SEQUENCE LISTING (2 sequences)

SEQ ID NO: 1 - DNA, 21 bp, Artificial Sequence
  Primer
aggagatcccagataaatgtg

SEQ ID NO: 2 - DNA, 20 bp, Artificial Sequence
  Primer
ctgtgcccatagcaactgag

(19) United States
(12) Patent Application Publication
(10) Pub. No.: US 2011/0086047 A1
(43) Pub. Date: Apr. 14, 2011

(54) FC RECEPTOR HOMOLOG ANTIBODIES AND USES THEREOF

(75) Inventors: Randall S. Davis, Birmingham, AL (US); Max D. Cooper, Birmingham, AL (US); Chuen-Miin Leu, Taipai City (TW); Goetz R. A. Ehrhardt, Birmingham, AL (US)

(73) Assignee: THE UAB RESEARCH FOUNDATION, Birmingham, AL (US)

(21) Appl. No.: 12/779,761

(22) Filed: May 13, 2010

Related U.S. Application Data
(63) Continuation of application No. 11/576,022, filed on May 30, 2007, now abandoned.
(60) Provisional application No. 60/613,908, filed on Sep. 27, 2004.

(51) Int. Cl. A61K 39/395; C07K 16/28; A61P 35/00; C12N 5/16; G01N 33/53

(52) U.S. Cl. 424/173.1; 530/387.1; 530/387.3; 530/388.15; 435/326; 435/7.1

(57) ABSTRACT

Provided herein are antibodies having the same epitope specificity as an antibody produced by the hybridoma cell line deposited with the American Type Culture Collection as hybridoma 4-2A6, 1-5A3, or 1-3B4. Further provided herein are methods of identifying and selecting cells that express FcRH1 or FcRH4. Methods of diagnosing and treating a subject with a malignancy of a hematopoietic cell lineage or an autoimmune disease and methods of modulating a humoral immune response in a subject are also provided herein. Further provided herein are polypeptides comprising one or more complementary determining regions of the disclosed antibodies and nucleic acids that encode the disclosed polypeptides.

### CROSS REFERENCE TO RELATED APPLICATIONS

This application claims the benefit of U.S. Provisional Application No. 60/613,908, filed Sep. 27, 2004, which is hereby incorporated herein by reference in its entirety.

### ACKNOWLEDGEMENTS

This invention was made with government support under Grant(s) AI39816 (MDC) and AI55638 (RSD) awarded by the National Institutes of Health. The government has certain rights in the invention.

### BACKGROUND OF THE INVENTION

Cancer is a common cause of death and morbidity in the United States and worldwide. Cancer is characterized by an increase in the number of abnormal, or neoplastic, cells derived from a normal tissue which proliferate to form a tumor mass, the invasion of adjacent tissues by these neoplastic tumor cells, and the generation of malignant cells that can eventually spread, or metastasize, via the blood or lymphatic system to other sites within the body.

Cancers that involve cells generated during hematopoiesis, a process by which cellular elements of blood, such as lymphocytes, leukocytes, platelets, erythrocytes and natural killer cells are generated, are referred to as hematopoietic cancers. In attempts to discover effective cellular targets for therapy of hematopoietic cancers, researchers have sought to identify transmembrane, or otherwise membrane-associated polypeptides that are specifically expressed on the surface of one or more types of cancer cells as compared to one or more normal non-cancerous cell.

Although, there has been some success in targeting such polypeptides with targeted therapy and for diagnosis, there is a great need for additional agents for diagnostic and therapeutic targeting of hematopoietic cancers. For example, B cell chronic lymphocytic leukemia (B-CLL) is the most common leukemia found among adults in Western countries with an estimated incidence of 4-7 per 100,000. This incurable disease is characterized by a progressive increase of anergic, self reactive, monoclonal B lineage cells that accumulate in the bone marrow and peripheral blood in a protracted fashion over many years or instead may adopt an aggressive course which eventually manifests as bulky infiltration of lymphoid organs, progressive cellular and humoral immunodeficiency, autoimmune disease, and hematologic impotence.

The clinical response to single agent immunotherapies such as Rituxan® (anti-CD20) and Campath® (anti-CD52), used to supplement the limited benefit of chemotherapy in these patients, has been inadequate. This is likely a function of the low level expression of CD20 frequently found on B-CLL cells and the broad expression of CD52 on T cells, macrophages, monocytes and eosinophils which results in increased toxicity and infections.

Given these therapeutic limitations, B-CLL patients would benefit from alternative targeted diagnostic and therapeutic reagents. Moreover, patients could benefit from targeted therapies and diagnostics for other malignancies including, but not limited to, diffuse large B-Cell lymphomas, follicular lymphomas, mantle cell lymphomas, mucosa-associated lymphoid tissue (MALT) lymphomas, multiple myeloma, and Waldenstrom's macroglobulinemia. Also needed are agents for diagnosing and treating autoimmune disease, and for modulating humoral immune responses.

### SUMMARY OF THE INVENTION

Provided herein are antibodies having the same epitope specificity as an antibody produced by the hybridoma cell line deposited with the American Type Culture Collection as hybridoma 4-2A6, 1-5A3, or 1-3B4. Further provided herein are methods of identifying and selecting cells that express FcRH1 or FcRH4. Methods of diagnosing and treating a subject with a malignancy of a hematopoietic cell lineage or an autoimmune disease and methods of modulating a humoral immune response in a subject are also provided herein. Further provided herein are polypeptides comprising one or more complementary determining regions of the disclosed antibodies and nucleic acids that encode the disclosed polypeptides.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate (one) several embodiment(s) of the invention and together with the description, serve to explain the principles of the invention.

FIGS. 1 (A and B) shows specificity of two anti-FcRH1 monoclonal antibodies. (A) Human 293T cells were transiently transfected with FcRH1-5 expression vectors and stained with 3B4 (thick grey line) or 5A3 (thick black line) monoclonal antibodies for immunofluorescence analysis. Cell surface expression of FcRH2-5 was confirmed by the use of specific monoclonal antibodies against each molecule. (B) Recombinant FcRH proteins and the chicken Ig-like receptor (CHIR) were immunoblotted with anti-FcRH1 (5A3).

FIG. 2 shows FcRH1 expression by peripheral blood mononuclear cells. Human blood mononuclear cells purified by Ficoll centrifugation were stained with biotinylated Fab fragment of 5A3 followed by streptavidin-APC and PE-conjugated antibodies to lineage specific markers: CD19+ B lineage cells, CD3⁺ T cells, CD14⁺ myeloid lineage cells, and CD56⁺ NK cells. Cells in the lymphocyte light-scatter gate were analyzed for CD19, CD3 and CD56, and cells in the myeloid gate are shown here for CD14 and FcRH1 staining. The same FcRH1 expression pattern was found for 10 donors of European, African, or Asian ancestry. Similar results were obtained with the 3B4 antibody.

FIGS. 3 (A and B) shows FcRH1 expression by B lineage cells in bone marrow and tonsils. (A) Bone marrow mononuclear cells from adult ribs were purified and 3-color immunofluorescent staining was performed. This pattern of FcRH1 expression by pre-B and B cells was confirmed for three additional bone marrow samples. (B) Tonsillar B cells purified using CD19 microbeads were stained with antibodies against CD38, IgD, and biotin-5A3 followed by streptavidin-APC. The different B cell subpopulations were gated for FcRH1 analysis based on their CD38 and IgD expression. Note the biphasic pattern of FcRH1 expression by the pre-germinal center subpopulation.

FIGS. 4 (A and B) shows comparative analysis of FcRH1 expression by different subpopulations of tonsillar B lineage cells. (A) Mean fluorescent intensity (MFI) of cell surface FcRH1 (±1 standard derivation) is shown for 7 individuals. (B) Tonsillar subpopulations were sorted and FcRH1 mRNA levels were examined by real-time RT-PCR and normalized to GAPDH expression. Mean levels (±standard derivations) are shown for each subpopulation in 3 tonsillar samples.

FIG. 5 shows correlation between FcRH1 levels with cell size, cell cycle status, and surface IgD, IgM, CD80, and CD86 expression in tonsillar B cells. Tonsillar B cells were purified as described below for 4-color immunofluorescent analysis. Neve, pre-GC, and GC populations were subdivided into the indicated R1-R8 subsets for analysis. DNA content analysis was conducted after cell fixation in 100% ethanol, treatment with RNase A, and staining with propidium iodide (40 μg/ml).

FIGS. 6 (A, B, C, and D) shows analysis of B cell activation by FcRH1 ligation. (A) Concomitant FcRH1 ligation enhances BCR-induced calcium flux. Daudi B cells were labeled with the calcium indicator dye Fluo-4 and, a reference dye, SNARF-1 and calcium levels evaluated by flow cytometry before and after stimulation. Thin black line, streptavidin crosslinker alone (20 μg/ml). Thick black line, biotinylated F(ab′)₂ fragments of goat anti-human μ HC (2 μg/ml) plus streptavidin. Thick grey line, biotinylated F(ab′)₂ fragments of goat anti-human μ HC (2 μg/ml), biotinylated Fab fragments of anti-FcRH1 (5A3) (1 μg/ml) and streptavidin. (B) Ligation induced FcRH1 tyrosine phosphorylation. HA-tagged FcRH1 overexpressing mouse IIA1.6 cells were serum-starved for 2 hr before incubation with biotinylated Fab fragments of anti-FcRH1 mAb plus streptavidin (20 μg/ml). Cell lysate (200 μg) was immunoprecipitated with anti-HA antibody and the immunoprecipitates immunoblotted with either anti-phosphotyrosine antibody (anti-pTyr) or anti-FcRH1 antibody. (C) FcRH1 ligation induces DNA synthesis. Purified tonsillar B cells were incubated in 96-well plates (10⁵/well) for 72 hr in the presence or absence of varying concentrations of biotinylated Fab fragments of anti-FcRH1 or control mAbs plus streptavidin (20 μg/ml). Cells were pulsed for an additional 16 hr with ³H-thymidine (1 μCi/well) before measuring ³H-thymidine incorporation. (D) FcRH1 co-ligation enhances BCR-induced B cell proliferation. Tonsillar B cells were incubated in 96-well plates (10⁵/well) for 72 hr in the presence or absence of anti-μ HC antibody (DA4.4, 1 μg/ml), biotinylated Fab fragments of anti-FcRH1 mAbs (3 μg/ml) plus streptavidin (20 μg/ml), or the combination of both antibodies. Cells were analyzed as in C.

FIG. 7 shows results demonstrating that FcRH1 does not bind to tested human Ig Isotypes. Transductants were generated as described by Ehrhardt et. al. 2003, using the BW5147 mouse T-cell line. Control and transduced cells were incubated with 12CA5 anti-HA (Roche) or with human IgA, IgM, IgG1, IgG2, IgG3, IgG4, or heat-aggregated IgG (100 μg/mL; Sigma-Aldrich, St. Louis, Mo.) before staining with PE-conjugated goat anti-human Ig and analysis by cell surface immunofluorescence.

FIGS. 8 (A and B) shows anti-FcRH4 monoclonal antibody 2A6 is specific for FcRH4. (A) A20-IIA1.6 cells were transiently transfected with expression constructs encoding a FcRH4-GFP fusion protein (filled histogram) or GFP-only control constructs (open histogram). The cells were stained using biotinylated F(ab′)₂-fragments of anti-FcRH4 antibodies and streptavidin-PE. For analysis, gates were set on the GFP-positive cell fraction of transfected cells. (B) Lysates from BOSC23 cells transiently transfected with the indicated GFP fusion constructs or empty vector control constructs were subjected to immunoprecipitation with anti-FcRH4 antibodies. The immunoprecipitates (top panel) and whole cell lysates (bottom panel) were separated by SDS-PAGE and probed with anti-GFP antibodies.

FIGS. 9 (A-F) shows FcRH4 is expressed predominantly on IgD/CD38⁻ memory B cells. (A) CD19-purified human tonsillar B cells were stained with anti-CD38, anti-IgD and anti-FcRH4. A sub-population stained positive for FcRH4 (gate M2). (B and C) Analysis of total tonsillar B cells (gate M1) and FcRH4-positive tonsillar B cells (gate M2) for expression of CD38 and IgD. (D and E) CD19-purified tonsillar B cells were stained for CD38, IgD, CD27 and FcRH4. The gate was set on the CD38−/IgD− memory B cell population and analyzed for forward scatter (FSC) versus FcRH4 (D) and CD27 versus FcRH4 (E). (F) GIEMSA stain of FcRH4-positive (top panel) and FcRH4-negative (bottom panel) memory B cells. (magnification x400).

FIG. 10 shows surface markers expressed by FcRH4-positive and FcRH4-negative tonsillar memory B cells. CD19-purified tonsillar B cells were stained for CD38, IgD, FcRH4 and the indicated cell surface markers. For analysis, the gate was set on the IgD−/CD38− memory B cell population. FcRH4-positive cells are indicated by a solid line and FcRH4-negative cells by a dashed line. The filled gray histogram indicates the isotype control stain.

FIGS. 11 (A and B) shows quantitative assessment of transcription factor and chemokine receptor mRNA expression by FcRH4⁺ and FcRH4⁻ memory B cells. (A) shows mRNA derived trom FcRH4⁺ and FcRH4⁻ memory B cells, germinal center (GC) B cells and plasma cells (PC). Transcripts of BLIMP-1, XBP-1, and IRF4 were barely detectable in either FcRH4⁺ and FcRH4⁻ memory B cells or in the germinal center B cells, whereas a prominent signal was observed for these transcripts in plasma cells. (B) mRNA from FcRH4⁺ and FcRH4⁻ memory B cells was used as template for real-time PCR analysis of the indicated genes. All values are normalized to expression levels of the large subunit of the RNA-polymerase 2. Values represent the mean±SEM from three independent cDNA preparations from three different tonsil samples, with each PCR performed in duplicate.

FIG. 12 (A and B) show expression of FcRH4 on multiple myeloma cells. (A) Quantitative PCR analysis of FcRH4 mRNA of the indicated cell lines. All values were normalized to expression of RP-2. Values represent mean±SD (n=4). (B) FACS analysis of NCI-H929, U226, and RPMI-8226 cells. Staining with an isotype matched control antibody is also shown.

FIG. 13 shows FcRH4-positive memory B cells respond to cytokine stimulation but not to ligation of the BCR. Purified FcRH4-positive (filled bars, ▪) and FcRH4-negative (open bars, □) memory B cells were cultured for 40 hours in the presence of the indicated stimuli. After addition of ³H-thymidine for an additional 10 hours the cells were harvested and ³H-thymidine incorporation assessed. Shown is a representative experiment out of at least 4 independently performed experiments. Data represent mean+/−SD.

FIG. 14 shows FcRH4-positive cells secrete increased amounts of immunoglobulins. (A) Supernatants from purified FcRH4-positive (filled bars, ▪) and FcRH4-negative (open bars, □) memory B cells were analyzed for secreted immunoglobulins by capture ELISA. The cells were treated with the indicated factors for 4 days. Shown is a representative experiment out of at least 4 independently performed experiments. Data represent mean+/−SD. (B) Elispot analysis of FcRH4-positive and FcRH4-negative memory B cells. The cells from (A) were plated onto Elispot plates coated with anti-human IgA, IgM or IgG. After incubation for 5 hours, the number of Ig-secreting cells was assessed.

FIG. 15 shows immunoprecipitation of FcRH1 from B-cell Chronic Lymphocytic Leukemia B-CLL cells.

FIG. 16 shows expression of FcRH1 in B-cell Chronic Lymphocytic Leukemia (B-CLL) cells.

FIG. 17 shows expression of FcRH1 in mantle cell lymphoma cells.

### DETAILED DESCRIPTION

The present invention may be understood more readily by reference to the following detailed description of the invention and the Examples included therein and to the Figures and their previous and following description.

In this specification and in the claims that follow, reference will be made to a number of terms that shall be defined to have the following meanings:

As used in the specification and the appended claims, the singular forms “a,” “an” and “the” include plural referents unless the context clearly dictates otherwise. Thus, for example, reference to a receptor includes mixtures of various receptors, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. For example, the phrase “optionally comprises all complementary determining regions” means that all complementary determining regions may or may not be present and that the description includes both the presence and absence of all complementary determining regions.

As used throughout, by “subject” is meant an individual. Preferably, the subject is a mammal such as a primate, and, more preferably, a human. The term “subject” can include domesticated animals, such as cats, dogs, etc., livestock (e.g., cattle, horses, pigs, sheep, goats, etc.), and laboratory animals (e.g., mouse, rabbit, rat, guinea pig, etc.).

Receptors for the Fc region (FcRs) of Igs have broad tissue distribution patterns and can modulate cellular and humoral immunity by linking their antibody ligands with effector cells of the immune system (Ravetch, J. V. & Kinet, J.-P. (1991) Annu. Rev. Immunol. 9, 457-492; Daeron, M. (1997) Annu. Rev. Immunol. 15, 203-234). These cellular receptors have the ability to sense humoral concentrations of antibody, initiate cellular responses in host defense, and participate in autoimmune disorders (Ravetch, J. V. & Bolland, S. (2001) Annu. Rev. Immunol. 19, 275-290). Their diverse regulatory roles depend on the Ig isotype specificity and cellular distribution of the individual FcR. These Ig superfamily members share similarities in their ligand binding subunits, and they may have inhibitory or activating signaling motifs in their intracellular domains or instead pair with signal transducing subunits possessing activating signaling motifs.

Receptors for the Fc portion of immunoglobulins (FcRs) may differentially modulate both cellular and humoral immune responses depending upon their Ig isotype binding specificity and the type of cells that bear them. (Ravetch, J. V. & Kinet, J.-P. (1991) Annu. Rev. Immunol. 9, 457-492; Daeron, M. (1997) Annu. Rev. Immunol. 15, 203-234). The FcR often pair with adaptor transmembrane proteins that possess immunoreceptor tyrosine-based activation motifs (ITAM) or, alternatively, may possess either ITAM or immunoreceptor tyrosine-based inhibitory motifs (ITIM) in their cytoplasmic domain. After FcR ligation by antibodies complexed with antigen, tyrosines in the ITAM or ITIM are phosphorylated by Src family kinases to engage SH2-containing molecules and other downstream signaling components in the cellular response cascade (Vely, F (1997) J. Immunol. 159, 2075-2077).

A recently identified family of Fc receptor homolog (FcRH) genes are located within the FcR locus on chromosome 1 in humans (Davis, R. S. (2001) Proc. Natl. Acad. Sci. 9772-9777). At the transcript level, the FcRH genes are differentially expressed by B lineage cells and overexpressed in some B cell malignancies. The predicted FcRH 1-5 transmembrane glycoproteins contain consensus ITIM, ITAM-like, or both types of motifs. FcRH1, has two ITAM-like motifs, an acidic glutamic acid residue in its transmembrane region, and three extracellular Ig-like domains.

FcRH4 is a recently identified member of a family of Ig-domain containing cell surface receptors with a high degree of similarity to classical Fc-receptors. In situ hybridization experiments, quantitative RT-PCR analysis and immunohistochemical experiments suggest an expression pattern restricted to memory B cells. Functional analysis of the intracellular domain of FcRH4 established this molecule as a potent inhibitor of B cell signaling.

Provided herein are antibodies selective for FcRH1 or selective for FcRH4. Thus, provided herein are antibodies having the same epitope specificity as an antibody produced by the hybridoma cell line 4-2A6, which was received for deposit with the American Type Culture Collection (ATCC), Manassas, Va., on Sep. 24, 2004 and assigned ATCC # PTA-6236. The description of the deposited material was “mouse hybridoma cell line Ag8 expressing mouse anti-human FcRH4-γ_{2aK} isotype,” with the strain designation 4-2A6 and the attorney docket number as 21085.0128U1. Also provided herein are antibodies having the same epitope specificity as an antibody produced by the hybridoma cell line 1-5A3, which was received for deposit with the American Type Culture Collection (ATCC), Manassas, Va., on Sep. 16, 2004 and assigned ATCC # PTA-6219. The description of the deposited material was “mouse hybridoma cell line Ag8 expressing mouse anti-human FcRH1-γ_{2bK} isotype,” with the strain designation 1-5A3 and the attorney docket number as 21085.012801. Further provided herein are antibodies having the same epitope specificity as an antibody produced by the hybridoma cell line 1-3B4 deposited Sep. 3, 2004 and assigned ATCC # PTA-6192. Optionally, the antibody is produced by a cell of the hybridoma cell line deposited with the American Type Culture Collection (ATCC) as hybridoma 4-2A6, 1-5A3, or 1-3B4. Further provided herein is a fragment of an antibody of the invention. Optionally, the antibody or fragment binds to an FcRH4 receptor molecule like antibodies made by cells of Hybridoma 4-2A6, or antibodies having the same epitope specificity. Optionally, the antibody or fragment activates or inhibits the FcRH4 receptor molecule. Optionally, the antibody or fragment binds to an FcRH1 receptor molecule like antibodies made by cells of Hybridoma 1-5A3 or Hybridoma 1-3B4, or antibodies having the same epitope specificity. Also provided herein is a molecular complex comprising an antibody or fragment of the invention and a therapeutic agent. Optionally, the antibody fragment is selected from the group consisting of Fv, Fab, Fab′ and F(ab′)2 fragments.

The antibodies and fragments thereof of the present invention can be utilized to modulate FcRH1 or FcRH4 receptor functions. An FcRH1 or an FcRH4 receptor can be modulated by activation or inhibition of the receptor. As utilized herein, activation means that the antibody or fragment thereof binds to FcRH1 or FcRH4 and effects one or more receptor functions normally associated with FcRH1 or FcRH4. Activation does not have to be complete as this can range from a slight increase in a receptor function to an increase similar or greater to that observed upon activation of FcRH1 or FcRH4 by its natural ligand. As utilized herein, inhibition means that the antibody binds to FcRH1 or FcRH4 and inhibits one or more receptor functions normally associated with FcRH1 or FcRH4. Inhibition does not have to be complete as this can range from a slight decrease in a receptor function to complete inhibition of a receptor function associated with FcRH1 or FcRH4. The antibodies or fragments thereof can also be used to block constitutive binding by the given receptor's ligand.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively. The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest” National Institutes of Health, Bethesda, Md. (1987)). The constant domains are not involved directly in binding an antibody to an antigen, but exhibit various effector functions, such as participation of the antibody in antibody-dependent cellular toxicity.

As used herein, the term “epitope” is meant to include any determinant capable of specific interaction with the antibodies of the invention. Epitopic determinants usually consist of chemically active surface groupings of molecules such as amino acids or sugar side chains and usually have specific three dimensional structural characteristics, as well as specific charge characteristics. Provided herein are fragments of FcRH1 and FcRH4 consisting of the portion of the FcRH1 or FcRH4 molecule bound by the antibodies and antibody fragments disclosed herein. Further provided herein are methods of modulating the activity of an FcRH1 or FcRH4 receptor molecule in a system comprising administering to a subject the fragment of FcRH1 or FcRH4 that is the portion bound by the antibodies and antibody fragments disclosed herein. By modulation is meant an activation or inhibition of an activity of FcRH1 or FcRH4 as described above. A system is meant to include both in vivo and in vitro systems. For example, a system may be a cell culture or a subject as defined herein.

The term “antibody or fragments thereof, or antibody fragment” can also encompass chimeric antibodies and hybrid antibodies, with dual or multiple antigen or epitope specificities, and fragments, such as F(ab′)₂, Fab′, Fab and the like, including hybrid fragments. Thus, fragments of the antibodies that retain the ability to bind their specific antigens are provided. For example, fragments of antibodies which maintain FcRH1 or FcRH4 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

In one embodiment, the antibody is a monoclonal antibody. The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired activity (See, U.S. Pat. No. 4,816,567 and Morrison et al., Proc. Natl. Acad. Sci. USA, 81:6851-6855 (1984)).

Further provided herein are hybridoma cells that produce an antibody having the same epitope specificity as an antibody produced by the hybridoma cell line deposited with the American Type Culture Collection (ATCC) as hybridoma 4-2A6 (ATCC # PTA-6236), 1-5A3 (ATCC # PTA-6219), or 1-3B4 (ATCC # PTA-6192). An example of such hybridoma cells is a hybridoma cell which produces a monoclonal antibody that specifically binds an epitope contained within FcRH1 or FcRH4. The present invention further provides a hybridoma cell which produces a monoclonal antibody that specifically binds an epitope contained within the extracellular portion of FcRH1 or FcRH4.

Monoclonal antibodies of the invention may be prepared using hybridoma methods, such as those described by Kohler and Milstein, Nature, 256:495 (1975) or Harlow and Lane, Antibodies, A Laboratory Manual. Cold Spring Harbor Publications, New York, (1988). In a hybridoma method, a mouse or other appropriate host animal, is typically immunized with an immunizing agent to elicit lymphocytes that produce or are capable of producing antibodies that will specifically bind to the immunizing agent. Alternatively, the lymphocytes may be immunized in vitro. Preferably, the immunizing agent comprises an FcRH. More preferably, the immunizing agent comprises FcRH1 or FcRH4 or an extracellular fragment thereof. More specifically, the immunizing agent can comprise the binding site of antibodies produced by cells of the hybridoma deposited with the ATCC as hybridoma 4-2A6, 1-5A3, or 1-3B4. Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding a portion of FcRH, preferably the extracellular region or selected epitope, is injected into the host animal according to methods known in the art. Optionally, a portion of FcRH1 or FcRH4, preferably the extracellular region or selected epitope, can be injected into the host animal according to methods known in the art.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent; such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63).

The culture medium in which the hybridoma cells are cultured can then be assayed for the presence of monoclonal antibodies directed against FcRH1 or FcRH4 or selected epitopes thereof. For example, the culture medium can be assayed for the presence of monoclonal antibodies directed against FcRH1 or FcRH4. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography. The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody comprising one antigen-combining site having specificity for FcRH1 or FcRH4 and another antigen-combining site having specificity for a different antigen.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)₂ fragment that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)₂ fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

An isolated immunogenically specific fragment of the antibody is also provided. A specific fragment of the antibody can be isolated from the whole antibody by chemical or mechanical disruption of the molecule. Similarly, fragments or epitopes of FcRH1 or FcRH4 are provided and can be obtained in a comparable way. The purified fragments or epitopes thus obtained can be tested to determine their immunogenicity and specificity by the methods taught herein. Immunoreactive fragments of the antibody can also be synthesized directly. An immunoreactive fragment is defined as an amino acid sequence of at least about two to five consecutive amino acids derived from the antibody amino acid sequence.

One method of producing proteins comprising the antibodies or polypeptides of the present invention is to link two or more peptides or polypeptides together by protein chemistry techniques. For example, peptides or polypeptides can be chemically synthesized using currently available laboratory equipment using either Fmoc (9-fluorenylmethyl-oxycarbonyl) or Boc (tert-butyloxycarbonoyl) chemistry. (Applied Biosystems, Inc., Foster City, Calif.). One skilled in the art can readily appreciate that a peptide or polypeptide corresponding to the antibody of the present invention, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W.H. Freeman and Co., N.Y. (1992); Bodansky M and Trost B., Ed. (1993) Principles of Peptide Synthesis. Springer-Verlag Inc., NY). Alternatively, the peptide or polypeptide can by independently synthesized in vivo as described above. Once isolated, these independent peptides or polypeptides may be linked to form an antibody or fragment thereof via similar peptide condensation reactions.

For example, enzymatic ligation of cloned or synthetic peptide segments can allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-α-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments can be chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

The invention also provides fragments of antibodies that have bioactivity. The polypeptide fragments of the present invention can be recombinant proteins obtained by cloning nucleic acids encoding the polypeptide in an expression system capable of producing the polypeptide fragments thereof, such as a bacterial, adenovirus or baculovirus expression system. For example, one can determine the active domain of an antibody from a specific hybridoma that can cause a biological effect associated with the interaction of the antibody with FcRH1 or FcRH4. For example, amino acids found to not contribute to either the activity or the binding specificity or affinity of the antibody can be deleted without a loss in the respective activity. Similarly, fragments of FcRH1 and FcRH4 are provided, wherein the fragments comprise the binding site for the antibodies produced by the hybridomas deposited with the ATCC as hybridoma 4-2A6, 1-5A3, or 1-3B4.

For example, amino or carboxy-terminal amino acids can be sequentially removed from either the native or the modified non-immunoglobulin molecule or the immunoglobulin molecule and the respective activity assayed in one of many available assays. In another example, a fragment of an antibody can comprise a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry of cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments of the invention, whether attached to other sequences, can also include insertions, deletions, substitutions, or other selected modifications of particular regions or specific amino acids residues, provided the activity of the fragment is not significantly altered or impaired compared to the nonmodified antibody or epitope. These modifications can provide for some additional property, such as to remove or add amino acids capable of disulfide bonding, to increase its bio-longevity, to alter its secretory characteristics, etc. In any case, the fragment can possess a bioactive property, such as binding activity, regulation of binding at the binding domain, etc. Functional or active regions may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

Further provided herein is a humanized or human version of the antibody. Optionally, the humanized or human version binds to an FcRH4 receptor molecule or to an FcRH1 receptor molecule. Optionally, the antibody activates or inhibits the FcRH4 receptor molecule or the FcRH1 receptor molecule.

Optionally, the humanized or human antibody comprises at least one complementarity determining region (CDR) of an antibody having the same epitope specificity as an antibody produced by the hybridoma cell line deposited with the ATCC as hybridoma 4-2A6, 1-5A3, or 1-3B4. For example, the antibody can comprise all complementarity determining regions (CDRs) of an antibody having the same epitope specificity as an antibody produced by the hybridoma cell line deposited with the ATCC as hybridoma 4-2A6, 1-5A3, or 1-3B4. Further provided herein is a molecular complex comprising the humanized or human antibody and a therapeutic agent. Optionally, the humanized or human antibody can comprise at least one residue of the framework region of the monoclonal antibody produced by the disclosed hybridoma cell line.

Humanized and human antibodies can be made using methods known to a skilled artesian for example, the human antibody can be produced using a germ-line mutant animal or by a phage display library.

Antibodies can also be generated in other species and “humanized” for administration to humans. Alternatively, fully human antibodies can also be made by immunizing a mouse or other species capable of making a fully human antibody (e.g., mice genetically modified to produce human antibodies) and screening clones that bind FcRH1 or FcRH4. See, e.g., Lonberg and Huszar (1995) Human antibodies from transgenic mice, Int. Rev. Immunol. 13:65-93, which is incorporated herein by reference in its entirety for methods of producing fully human antibodies. As used herein, the term “humanized” and “human” in relation to antibodies, relate to any antibody which is expected to elicit a therapeutically tolerable weak immunogenic response in a human subject. Thus, the terms include fully humanized or fully human as well as partially humanized or partially human.

Humanized forms of non-human (e.g., murine) antibodies are chimeric immunoglobulins, immunoglobulin chains or fragments thereof (such as Fv, Fab, Fab′, F(ab′)₂, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all or at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The nucleotide sequences encoding the monoclonal antibodies of the present invention can be readily isolated and sequenced using conventional procedures (e.g., by using oligonucleotide probes that are capable of binding specifically to genes encoding the heavy and light chains of murine antibodies). These nucleotide sequences can also be modified, or humanized, for example, by substituting the coding sequence for human heavy and light chain constant domains in place of the homologous murine sequences (See U.S. Pat. No. 4,816,567 which is incorporated herein in its entirety by this reference). The nucleotide sequences encoding any of the humanized antibodies of the present invention can be expressed in appropriate host cells. These include prokaryotic host cells including, but not limited to, E. coli, Bacillus subtilus, other enterobacteriaceae such as Salmonella typhimurium or Serratia marcesans, and various Pseudomonas species. Eukaryotic host cells can also be utilized. These include, but are not limited to yeast cells (for example, Saccharomyces cerevisiae and Pichia pastoris), and mammalian cells such as VERO cells, HeLa cells, Chinese hamster ovary (CHO) cells W138 cells, BHK cells, COS-7 cells, 293T cells and MDCK cells. The antibodies produced by these cells can be purified from the culture medium and assayed for binding, activity, specificity or any other property of the monoclonal antibodies by utilizing the teaching set forth herein and methods standard in the art.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679).

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

In one embodiment, the antibody or fragment thereof is a single chain antibody. In another embodiment, the antibody or fragment is labeled. Optionally the antibody or fragment is conjugated or fused with a therapeutic agent or fragment thereof.

As disclosed herein, an antibody or fragments thereof that bind to the extracellular domain of the FcRH1 or FcRH4 receptor may be linked to a therapeutic agent, thereby forming a molecular complex. For example, the complex could be designed to target FcRH1 or FcRH4 positive cells and cause a desired physiologic effect including, for example, cell death or stasis. The linkage is preferably covalent, but can also be noncovalent (e.g., ionic). Therapeutic agents include but are not limited to toxins, including but not limited to plant and bacterial toxins, small molecules, peptides, polypeptides and proteins. Genetically engineered fusion proteins, in which genes encoding for an antibody or fragments thereof, including the Fv region, can be fused to the genes encoding a toxin to deliver a toxin to the target cell are also provided. As used herein, a “target cell” or “target cells” are FcRH1 or FcRH4 positive cells, including for example, malignant cells of hematopoietic cell lineage, or activated or inactivated B cells. Non-lymphoid cells like myeloid cells can also be transformed to express FcRH1 or FcRH4.

Other examples of therapeutic agents include chemotherapeutic agents, a radiotherapeutic agent, and immunotherapeutic agent, as well as combinations thereof. In this way, the “drug” (i.e., the molecular complex) delivered to the subject can be multifunctional, in that it exerts one therapeutic effect by binding to the extracellular domain of FcRH1 or FcRH4 and a second therapeutic by delivering a supplemental therapeutic agent. Binding of a monoclonal antibody to the FcRH1 or FcRH4 receptor can cause internalization of the receptor, which is useful for introducing a therapeutic agent such as a toxin into a cancer cell.

It should be understood that the invention is not limited by the nature of the therapeutic agent linked to the antibody or fragment; any therapeutic agent which is intended for delivery to the target cell can be complexed to the antibody of the invention. The therapeutic agent can act extracellularly, for example by initiating or affecting an immune response, or it can act intracellularly, either directly by translocating through the cell membrane or indirectly by, for example, affecting transmembrane cell signaling. The therapeutic agent is optionally cleavable from the antibody or fragment. Cleavage can be autolytic, accomplished by proteolysis, or affected by contacting the cell with a cleavage agent. Moreover, the antibody or fragments thereof can also act extracellularly, for example by initiating, affecting, enhancing or reducing an immune response without being linked in a molecular complex with a therapeutic agent. Such an antibody is known in the art as an “unconjugated” antibody. An unconjugated antibody can directly induce negative growth signal or apoptosis or indirectly activate a subject's defense mechanism to mediate anti-tumor activity. The antibody or fragment can be modified to enhance antibody-dependent cell killing. For example, amino acid substitutions can be made in the Fc region of the antibodies or fragments disclosed herein to increase binding of Fc receptors for enhanced antibody dependent cell cytotoxicity or increased phagocytosis. The antibody or fragment can also be used to induce cell proliferation. By inducing cell proliferation, the effects of a chemotherapeutic or radiotherapeutic agent described herein can be enhanced.

Examples of toxins or toxin moieties include diphtheria, ricin, streptavidin, and modifications thereof. An antibody or antibody fragment may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The therapeutic moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such a therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

Further provided herein is an antibody or fragment, wherein the antibody or fragment is labeled with a detectable moiety or marker. The detectable marker can be any marker known to those skilled in the art, or as described herein. Optionally, the detectable marker is selected from the group consisting of a fluorescent moiety, an enzyme linked moiety, a biotinylated moiety and a radiolabeled moiety. By “label” or “detectable moiety” is meant any detectable tag that can be attached directly (e.g., a fluorescent molecule integrated into a polypeptide or nucleic acid) or indirectly (e.g., by way of binding to a primary antibody with a secondary or tertiary antibody with an integrated fluorescent molecule) to the molecule of interest. Thus, a “label” or “detectable moiety” is any tag that can be visualized with imaging methods. The detectable tag can be a radio-opaque substance, a radiolabel, a fluorescent label, or a magnetic label. The detectable tag can be selected from the group consisting of gamma-emitters, beta-emitters, alpha-emitters, positron-emitters, X-ray-emitters and fluorescence-emitters suitable for localization. Suitable fluorescent compounds include fluorescein sodium, fluorescein isothiocyanate, phycoerythrin, and Texas Red sulfonyl chloride. See, de Belder & Wik (Preparation and properties of fluorescein-labeled hyaluronate. Carbohydr. Res. 44(2):251-57 (1975). Those skilled in the art will know, or will be able to ascertain with no more than routine experimentation, other fluorescent compounds that are suitable for labeling the molecule.

Provided herein is a polypeptide comprising one or more complementarity determining regions (CDRs) of an antibody having the same epitope specificity as an antibody produced by the hybridoma cell line deposited with the ATCC as hybridoma 4-2A6, 1-5A3, or 1-3B4. Optionally, the polypeptide comprises one or more complementarity determining regions (CDRs) of the antibody with one or more conservative amino acid substitutions. Also provided herein are nucleic acids encoding the polypeptides.

There are numerous variations of the polypeptides that are contemplated, which include the presence or absence of conservative amino acid substitutions. Amino acid sequence modifications fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about 2 to 6 residues are deleted at any one site within the protein molecule. These variants ordinarily are prepared by site-specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known and include, for example, M13 primer mutagenesis and PCR mutagenesis. Amino acid substitutions are typically of single residues but may include multiple substitutions at different positions; insertions usually will be on the order of about from 1 to 10 amino acid residues but can be more; and deletions will range about from 1 to 30 residues, but can be more. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with Table 1 and are referred to as conservative substitutions.

TABLE 1
Amino Acid Substitutions

| Original Residue | Exemplary Substitutions |
|---|---|
| Ala | Ser |
| Arg | Lys |
| Asn | Gln |
| Asp | Glu |
| Cys | Ser |
| Gln | Asn |
| Glu | Asp |
| Gly | Pro |
| His | Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 1, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions that in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl. Modifications in the FcRH can also include modifications in glycosylation. In all mutational events, it is understood that the controlling aspect of the mutation is the function that the subsequent protein possesses. The preferred mutations are those that do not detectably change the desired function or that increase the desired function.

There are a variety of molecules disclosed herein that are nucleic acid based, including for example the nucleic acids that encode, for example, polypeptide comprising one or more complementarity determining regions (CDRs) of an antibody having the same epitope specificity as an antibody produced by the hybridoma cell line deposited with the ATCC as hybridoma 4-2A6, 1-5A3, or 1-3B4, as well as any other proteins, antibodies and fragments thereof disclosed herein. The disclosed nucleic acids are made up of for example, nucleotides, nucleotide analogs, or nucleotide substitutes. Non-limiting examples of these and other molecules are discussed herein.

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is a pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to one of the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

The term “nucleotide analog” refers to molecules that can be used in place of naturally occurring bases in nucleic acid synthesis and processing, preferably enzymatic as well as chemical synthesis and processing, particularly modified nucleotides capable of base pairing. This term includes, but is not limited to, modified purines and pyrimidines, minor bases, convertible nucleosides, structural analogs of purines and pyrimidines, labeled, derivatized and modified nucleosides and nucleotides, conjugated nucleosides and nucleotides, sequence modifiers, terminus modifiers, spacer modifiers, and nucleotides with backbone modifications, including, but not limited to, ribose-modified nucleotides, phosphoramidates, phosphorothioates, phosphonamidites, methyl phosphonates, methyl phosphoramidites, methyl phosphonamidites, 5′-β-cyanoethyl phosphoramidites, methylenephosphonates, phosphorodithioates, peptide nucleic acids, achiral and neutral internucleotidic linkages and normucleotide bridges such as polyethylene glycol, aromatic polyamides and lipids. Optionally, nucleotide analog is a synthetic base that does not comprise adenine, guanine, cytosine, thymidine, uracil or minor bases. These and other nucleotide and nucleoside derivatives, analogs and backbone modifications are known in the art (e.g., Piccirilli J. A. et al. (1990) Nature 343:33-37; Sanghvi et al (1993) In: Nucleosides and Nucleotides as Antitumor and Antiviral Agents, (Eds. C. K. Chu and D. C. Baker) Plenum, New York, pp. 311-323; Goodchild J. (1990) Bioconjugate Chemistry 1:165-187; Beaucage et al. (1993) Tetrahedron 49:1925-1963).

It is also possible to link other types of molecules to nucleotides or nucleotide analogs to make conjugates that can enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556).

The term “polynucleotide” is used broadly herein to mean a sequence of two or more deoxyribonucleotides or ribonucleotides that are linked together by a phosphodiester bond. As such, the term “polynucleotide” includes RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the term “polynucleotide” as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR). In various embodiments, a polynucleotide of the invention can contain nucleoside or nucleotide analogs, or a backbone bond other than a phosphodiester bond. In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., Nucl. Acids Res. 22:5220-5234 (1994); Jellinek et al., Biochemistry 34:11363-11372 (1995); Pagratis et al., Nature Biotechnol. 15:68-73 (1997)).

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995).

For example, the nucleic acids, such as those encoding the polypeptides comprising one or more complementarity determining regions (CDRs) of an antibody having the same epitope specificity as an antibody produced by the hybridoma cell line deposited with the ATCC as hybridoma 4-2A6, 1-5A3, or 1-3B4 can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986 (1994); Ecker and Crooke, BioTechnology 13: 351360 (1995), each of which is incorporated herein by reference). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

The disclosed nucleic acids include all degenerate sequences related to a specific polypeptide sequence, i.e. all nucleic acids having a sequence that encodes one particular polypeptide sequence as well as all nucleic acids, including degenerate nucleic acids, encoding the disclosed variants and derivatives of the polypeptide sequences.

Provided herein is a method of diagnosing a malignancy of hematopoietic cell lineage in a subject, comprising (a) contacting a biological sample of the subject with an antibody having the same epitope specificity as an antibody produced by the hybridoma cell line deposited with the ATCC as hybridoma 4-2A6, 1-5A3, or 1-3B4, or a fragment thereof under conditions that allow the antibody or fragment to bind to FcRH4 or FcRH1 in the biological sample, and (b) detecting binding by the antibody or fragment. Detecting can be performed using, for example, flow cytometry, or staining of preserved tissue sections. Other imaging modalities can also be used to detect the antibody or fragment. For example, ultrasound, computed tomography, optical coherence tomography, radiography, fluorescent detection and other modalities can be used if an appropriate contrast ligand is attached to the antibody or fragment thereof. Changes in the binding level or distribution as compared to binding in a control sample indicating a malignancy of hematopoietic cell lineage in the subject. Optionally, the malignancy of hematopoietic cell lineage is a malignancy of B cell lineage or is a malignancy of T cell lineage.

As used herein, changes in binding refer to changes in the amount or pattern (distribution) of binding. As used throughout, a “control sample” can comprise either a sample obtained from a control subject (e.g., from the same subject before treatment, or from a second subject without cancer, autoimmune, or inflammatory disease, or without treatment) or can comprise a known standard.

As used herein, the phrase “selectively binds,” “specific binding affinity,” or “selective for” refers to a binding reaction which is determinative of the presence of FcRH1 or FcRH4 in a heterogeneous population of proteins, cells, proteoglycans, and other biologics. Thus, under designated conditions, the antibodies or fragments thereof of the present invention bind to FcRH1, FcRH4, or protein core, epitope, fragment, or variant thereof and do not bind in a significant amount to other proteins or proteoglycans present in the subject, or in a biological sample as described herein.

Selective binding to an antibody under such conditions may require an antibody that is selected for its specificity for FcRH1, FcRH4 or a fragment thereof. A variety of immunoassay formats may be used to select antibodies that selectively bind with FcRH1, FcRH4, or a fragment thereof. For example, solid-phase ELISA immunoassays are routinely used to select antibodies selectively immunoreactive with a protein, proteoglycan, or variant, fragment, epitope, or protein core thereof. See Harlow and Lane. Antibodies, A Laboratory Manual. Cold Spring Harbor Publications, New York, (1988), for a description of immunoassay formats and conditions that could be used to determine selective binding. The binding affinity of a monoclonal antibody can, for example, be determined by the Scatchard analysis of Munson et al., Anal. Biochem., 107:220 (1980).

Preferably, in an ELISA, the binding of the antibody or fragments thereof of the present invention to FcRH1 or FcRH4 is at least 1.5 times the background level (i.e., comparable to non-specific binding or slightly above non-specific binding). More preferably, the binding of the antibody or fragments thereof of the present invention to FcRH1 or FcRH4 is at least 2.5 times the background level.

Provided herein is a method of identifying a hematopoietic cell that expresses FcRH4, or, in the alternative, FcRH1, in vitro, comprising contacting a biological sample with an antibody having the same epitope specificity as an antibody produced by the hybridoma cell line deposited with the ATCC as hybridoma 4-2A6, 1-5A3, or 1-3B4, or a fragment thereof under conditions that allow the antibody or fragment to bind to FcRH4 or FcRH1 in the sample detecting the binding of the antibody or fragment, the binding identifying a hematopoietic cell that expresses FcRH4 or FcRH1.

Further provided herein is a method of selecting a hematopoietic cell that expresses FcRH4 or FcRH1 or a purified population of hematopoietic cells that express FcRH4 or FcRH1, comprising contacting a biological sample with an antibody having the same epitope specificity as an antibody produced by the hybridoma cell line deposited with the ATCC as hybridoma 4-2A6, 1-5A3, or 1-3B4, or a fragment thereof under conditions that allow the antibody or fragment to bind to FcRH4 or FcRH1 expressing cells in the biological sample and selecting the cell or cells that bind the antibody or fragment, the selected cells being a hematopoietic cell that expresses FcRH4 or FcRH1.

As used herein “selecting” includes isolating or purifying or isolating a particular cell that expresses FcRH1 or FcRH4. Therefore, the antibodies or fragments can be used to isolate or purify any cell that expresses FcRH1 or FcRH4, including cells of lymphoid and non-lymphoid origin. For example, in the present instance, the antibodies or fragments can be used to isolate or purify hematopoietic cells that express FcRH1 or FcRH4. By way of non-limiting example, the disclosed compositions and methods can be used to select, purify, or isolate one or more B-cell chronic lymphocyte leukemia (B-CLL) cells, multiple myeloma cells, mantle cell lymphoma cells, MALT lymphoma cells, diffuse large B-cell lymphoma cells, follicular lymphoma cells, Waldenstrom's macroglobulinemia cells and/or any other malignant cell expressing FcRH1 or FcRH4. By selecting, purifying, or isolating cells expressing FcRH1 or FcRH4, malignant cells expressing these polypeptides can be identified and removed from a subject. For example, a population of cells comprising normal and malignant cells can be taken from the subject. Within the population cells, those expressing FcRH1 or FcRH4 can be identified, and selected for removal from the population, thereby leaving the population with a reduced number of FcRH1 or FcRH4 expressing cells. In this way, the number of malignant cells in the population expressing FcRH1 or FcRH4 can be reduced and the population with fewer malignant cells can be administered back to the subject.

Provided herein is a method of treating a subject with a malignancy of a hematopoietic cell lineage, comprising administering to the subject a therapeutically effective amount of an antibody having the same epitope specificity as an antibody produced by the hybridoma cell line deposited with the ATCC as hybridoma 4-2A6, 1-5A3, or 1-3B4, or a fragment thereof. Optionally, in the various methods of the invention, molecular complexes can be used instead of, or in addition to, an antibody or antibody fragment. Thus for example, the malignant cell of the subject is contacted with a therapeutically effective amount of a molecular complex comprising the antibody or fragment thereof and a therapeutic agent.

All of the methods disclosed herein can be utilized to treat any condition associated with changes in FcRH1 or FcRH4 receptor function. Such changes include changes in binding characteristics, changes in expression and changes in activity. Thus, any condition found to be associated with changes in FcRH1 or FcRH4 receptor function is a condition for which FcRH1 or FcRH4 is a therapeutic target.

Provided herein is a method of diagnosing an autoimmune or inflammatory disease in a subject, comprising contacting a biological sample of the subject with an antibody having the same epitope specificity as an antibody produced by the hybridoma cell line deposited with the ATCC as hybridoma 4-2A6, 1-5A3, or 1-3B4, or a fragment thereof under conditions that allow the antibody or fragment to bind to FcRH4 or FcRH1 in the biological sample and detecting binding by the antibody or fragment, changes in the antibody binding as compared to binding in a control sample indicating an autoimmune or inflammatory disease in the subject. For example, the disclosed methods can be used to diagnose inflammatory conditions such as infectious diseases.

Provided herein is a method of treating an autoimmune disease in a subject, comprising contacting, with a therapeutically effective amount of an antibody having the same epitope specificity as an antibody produced by the hybridoma cell line deposited with the ATCC as hybridoma 4-2A6, 1-5A3, or 1-3B4, or a fragment thereof, one or more FcRH4 or FcRH1 expressing cells of the subject.

Provided herein is a method of targeting B cells in a subject comprising administering to the subject an antibody having the same epitope specificity as an antibody produced by the hybridoma cell line deposited with the ATCC as hybridoma 4-2A6, 1-5A3, or 1-3B4, or a fragment thereof. Optionally, one or more therapeutic agent that binds the antibody or fragment thereof is administered to the subject. Thus, the B cell can be targeted, wherein the B cell is FcRH1 or FcRH4 positive, by administering an antibody or a fragment thereof and a therapeutic agent. The antibody or fragment thereof and the therapeutic agent can be administered separately or as a molecular complex. Such coupling of the antibody or fragment with the therapeutic agent as a molecular complex can be achieved by making an immunoconjugate or by making a fusion protein, comprising the antibody or antibody fragment and the one or more therapeutic agent.

The invention provides uses of the reagents described herein in in vitro and in vivo methods of diagnosing and treating a malignancy of hematopoietic cell lineage or an autoimmune disease in a subject. The reagents of the present invention are also useful in screening for disease manifestations. Such screening may be useful even before the onset of other clinical symptoms and could be used to screen subjects at risk for disease, so that prophylactic treatment can be started before the manifestation of other signs or symptoms.

By “malignancy” is meant a tumor or neoplasm whose cells possess one or more nuclear or cytoplasmic abnormalities, including, for example, high nuclear to cytoplasmic ratio, prominent nucleolar/nucleoli variations, variations in nuclear size, abnormal mitotic figures, or multinucleation. Malignancy may also refer to a tumor or neoplasm whose cells display abnormal growth, inhibition, or other abnormal behavioral characteristics common to malignant cells. These malignancies can be of lymphoid or non-lymphoid origin. “Malignancies of hematopoietic cell lineage” include, but are not limited to, myelomas, leukemias, lymphomas (Hodgkin's and non-Hodgkin's forms), T-cell malignancies, B-cell malignancies, and lymphosarcomas or other malignancies described in the REAL classification system or the World Health Organization Classification of Hematologic Malignancies. By way of non-limiting example, the disclosed compositions and methods can be used to treat or diagnose any malignancy comprising cells that express FcRH1 or FcRH4, such as a melanoma expressing FcRH1 or FcRH4. The disclosed compositions and methods can also be used to treat or diagnose B-cell chronic lymphocytic leukemia (B-CLL), multiple myeloma, mantle cell lymphoma, MALT lymphomas, diffuse large B-cell lymphomas, follicular lymphomas, and Waldenstrom's macroglobulinemia.

It should be noted that the absence or presence of specific FcRHs can be diagnostic for a particular malignancy of hematopoietic cell linage or can be diagnostic for a particular form of a malignancy (e.g., a specific form of leukemia).

“Inflammatory and autoimmune diseases” include, but are not limited to, systemic lupus erythematosus, Hashimoto's disease, rheumatoid arthritis, graft-versus-host disease, Sjögren's syndrome, pernicious anemia, Addison disease, scleroderma, Goodpasture's syndrome, Crohn's disease, autoimmune hemolytic anemia, sterility, myasthenia gravis, multiple sclerosis, Basedow's disease, thrombocytopenic purpura, insulin-dependent diabetes mellitus, allergy; asthma, atopic disease; arteriosclerosis; myocarditis; cardiomyopathy; glomerular nephritis; hypoplastic anemia; rejection after organ transplantation and numerous malignancies of lung, prostate, liver, ovary, colon, cervix, lymphatic and breast tissues.

Specifically, the diagnostic methods comprise the steps of contacting a biological sample of the subject with an antibody or fragment of the invention under conditions that allow the antibody or fragment to bind to cells of hematopoietic cell lineage and detecting the amount and/or pattern of binding. A change in the binding as compared to binding in a control sample indicates a malignancy or an inflammatory or autoimmune disease. Changes in the amount or pattern of binding either increased or decreased as compared to binding in a control sample indicate a malignancy or an inflammatory or autoimmune disease. For example, an over-expression of FcRH4 or FcRH1 may be detected in certain B cell malignancies including acute lymphocytic leukemias, non-Hodgkins lymphomas, aggressive or indolent lymphoproliferative disorders, such as MALT lymphomas, CLL, plasmacytoid lymphomas, follicular lymphomas, mantle cell lymphomas, diffuse large cell lymphomas, multiple myeloma, and Hodgkins lymphomas. Determination of binding changes is not, however, intended to be limited to these malignancy types. Changes in the amount or pattern of binding in other malignancy types could be readily determined when compared to a control population using methods known in the art. (Alizadeh et al., (2000) Nature 430, 503-511). Other changes in binding that can occur include an absolute increase in binding relative to the control population. Such an increase can indicate an increased number of malignant cells in a given biological sample when compared to a control. Similarly, an absolute increase, or decrease, in binding compared to a control may be detected in autoimmune or other inflammatory diseases.

The detecting step of the diagnostic method can be selected from methods routine in the art. For example, the detection step can be performed in vivo using a noninvasive medical technique such as radiography, fluoroscopy, sonography, imaging techniques such as magnetic resonance imaging, and the like. Thus, for example, a disclosed antibody or fragment thereof, can be labeled for detection in a subject using an appropriate imaging modality. If, for example, an antibody is radiolabeled then it can be detected using radiology. Similarly, if an antibody is labeled fluorescently, then it can be detected with a light sensitive detector. In vitro detection methods can be used to detect bound antibody or fragment thereof in an ELISA, RIA, immunohistochemically, flow cytometry, FACS, IHC, FISH, proteonomic arrays, or similar assays. The antibody, or fragment thereof, can be linked to a detectable label either directly or indirectly through use of a secondary and/or tertiary antibody; thus, bound antibody, fragment or molecular complex can be detected directly in an ELISA or similar assay.

As used throughout, “biological sample” refers to a sample from any organism. The sample can be, but is not limited to, peripheral blood, plasma, urine, saliva, gastric secretion, feces, bone marrow specimens, primary tumors, embedded tissue sections, frozen tissue sections, cell preparations, cytological preparations, exfoliate samples (e.g., sputum), fine needle aspirations, amnion cells, fresh tissue, dry tissue, and cultured cells or tissue. It is further contemplated that the biological sample of this invention can also be whole cells or cell organelles (e.g., nuclei). A biological sample can also include a partially purified sample, cell culture, or a cell line.

The sample can be unfixed or fixed according to standard protocols widely available in the art and can also be embedded in a suitable medium for preparation of the sample. For example, the sample can be embedded in paraffin or other suitable medium (e.g., epoxy or acrylamide) to facilitate preparation of the biological specimen for the detection methods of this invention. Furthermore, the sample can be embedded in any commercially available mounting medium, either aqueous or organic.

The sample can be on, supported by, or attached to, a substrate which facilitates detection. A substrate of the present invention can be, but is not limited to, a microscope slide, a culture dish, a culture flask, a culture plate, a culture chamber, ELISA plates, as well as any other substrate that can be used for containing or supporting biological samples for analysis according to the methods of the present invention. The substrate can be of any material suitable for the purposes of this invention, such as, for example, glass, plastic, polystyrene, mica and the like. The substrates of the present invention can be obtained from commercial sources or prepared according to standard procedures well known in the art.

Conversely, an antibody or fragment thereof, an antigenic fragment of FcRH1 or FcRH4 proteins, or polypeptide, or nucleic acid of the invention can be on, supported by, or attached to a substrate which facilitates detection. Such a substrate can include a chip, a microarray or a mobile solid support. Thus, provided by the invention are substrates including one or more of the antibodies or antibody fragments, antigenic fragments of FcRH1 or FcRH4 proteins, or nucleic acids of the invention,

The invention also provides a method of treating a malignancy of hematopoietic cell lineage or an inflammatory or autoimmune disease in a subject, comprising contacting the subject's malignant cells or inflammatory cells with a therapeutically effective amount of a reagent (e.g., an antibody or nucleic acid) or a therapeutic composition of a reagent of the invention. The reagent can be an antibody or a molecular complex as described herein. In one aspect, an antibody linked to biotin as described herein can be administered to a subject. Following administration of the biotin linked antibody, a streptavidin toxin can be administered. One example of such a streptavidin toxin is the biotinylated anti-CD20 interaction with a radiolabeled streptavidin complex known as zevalin. This type of complex could be generated with other small interactive compounds with the second agent containing a toxin, chemotherapeutic, immunotherapeutic compound binding to the antibody which is directly bound to cells.

The contacting step can occur by administration of the reagent or composition using any number of means available in the art. Typically, the reagent or composition is administered to the subject transdermally (e.g., by a transdermal patch or a topically applied cream, ointment, or the like), orally, subcutaneously, intrapulmonarily, transmucosally, intraperitoneally, intravascularly, intrauterinely, sublingually, intrathecally, intramuscularly, intraarticularly, etc. using conventional methods. In addition, the reagent or composition can be administered via injectable depot routes such as by using 1-, 3-, or 6-month depot injectable or biodegrable materials and methods. An antibody of the present invention or fragment thereof can be administered to an individual in combination (e.g., in the same formulation or in separate formulations) with another therapeutic agent (“combination therapy”). An antibody can be administered in a mixture with another therapeutic agent or can be administered in a separate formulation before, after, or simultaneously with the other therapeutic agent. When administered in separate formulations, an antibody and another therapeutic agent can be administered substantially simultaneously (e.g., within about 60 minutes, about 50 minutes, about 40 minutes, about 30 minutes, about 20 minutes, about 10 minutes, about 5 minutes, or about 1 minute, or less, of each other) or separated in time by about 1 hour, about 2 hours, about 4 hours, about 6 hours, about 10 hours, about 12 hours, about 24 hours, about 36 hours, or about 72 hours, or more.

The antibodies of the present invention or fragments thereof may also be administered in combination with effective amounts of one or more other therapeutic agents and/or in conjunction with radiation treatment. Thus, for example the antibodies of fragments thereof can be administered in combination with other immunotherapeutic agents. Such combination therapy can be used to treat a malignancy, an autoimmune condition, an immunodeficiency or to induce immunosupression. Therapeutic agents contemplated include chemotherapeutics, antibodies as well as immunoadjuvants and cytokines. Chemotherapies contemplated by the invention include chemical substances or drugs which are known in the art and are commercially available, such as Doxorubicin, 5-Fluorouracil, Cytosine arabinoside (“Ara-C”), Cyclophosphamide, Thiotepa, Busulfan, Cytoxin, Taxol, Methotrexate, Cisplatin, Melphalan, Vinblastine and Carboplatin. The antibodies or fragments thereof may be administered sequentially or concurrently with the one or more other therapeutic agents. The amount of antibody or fragment thereof and therapeutic agent depend, for example, on what type of therapeutic agents are used, the condition being treated, and the scheduling and routes of administration but would generally be less than if each were used individually.

Regardless of the route of administration, the amount of the reagent administered or the schedule for administration will vary among individuals based on age, size, weight, condition to be treated, mode of administration, and the severity of the condition. One skilled in the art will realize that dosages are best optimized by the practicing physician and methods for determining dosage are described, for example in Remington's Pharmaceutical Science, latest edition. Guidance in selecting appropriate doses for antibodies is found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical dose of the antibody used alone might range from about 1 μg/kg to up to 100 mg/kg of body weight or more per day, and preferably μg/kg to up to 1 mg/kg, depending on the factors mentioned above. An intravenous injection of the antibody or fragment thereof, for example, could be 10 ng-1 g of antibody or fragment thereof, and preferably 10 ng-1 mg depending on the factors mentioned above. For local injection, a typical quantity of antibody ranges from 1 μg to 1 mg. Preferably, the local injection would be at an antibody concentration of 1-100 μg/ml, and preferably 1-20 μg/ml.

The invention further provides a therapeutic composition of the reagent of the invention. Such a composition typically contains from about 0.1 to 90% by weight (such as 1 to 20% or 1 to 10%) of a therapeutic agent of the invention in a pharmaceutically acceptable carrier. Solid formulations of the compositions for oral administration may contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, without limitation, microcrystalline cellulose, corn starch, sodium starch, glycolate, and alginic acid. Tablet binders that may be used include acacia, methylcellulose, sodium carboxymethylcellulose, polyvinylpyrrolindone (Povidone™), hydroxypropyl methylcellulose, sucrose, starch, and ethylcellulose. Lubricants that may be used include magnesium stearates, stearic acid, silicone fluid, talc, waxes, oils, and colloidal silica.

Liquid formulations for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation into the lungs of the mammal to be treated.

Injectable formulations of the compositions may contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol, and the like). For intravenous injections, water soluble version of the compounds may be administered by the drip method, whereby a pharmaceutical formulation containing the antifungal agent and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as water-for-injection, 0.9% saline, or 5% glucose solution. A suitable insoluble form of the compound may be prepared and administered as a suspension in an aqueous base or a pharmaceutically acceptable oil base, such as an ester of a long chain fatty acid (e.g., ethyl oleate).

A topical semi-solid ointment formulation typically contains a concentration of the active ingredient from about 1 to 20%, e.g., 5 to 10%, in a carrier such as a pharmaceutical cream base. Various formulations for topical use include drops, tinctures, lotions, creams, solutions, and ointments containing the active ingredient and various supports and vehicles. The optimal percentage of the therapeutic agent in each pharmaceutical formulation varies according to the formulation itself and the therapeutic effect desired in the specific pathologies and correlated therapeutic regimens.

The effectiveness of the method of treatment can be assessed by monitoring the patient for known signs or symptoms of the conditions being treated. For example, in the treatment of a malignancy of hematopoietic cell lineage, the reduction or stabilization of the number of abnormally proliferative cells would indicate successful treatment. In the treatment of arthritis, for example, a reduction in the amount of joint inflammation would indicate successful treatment. Thus, by “therapeutically effective” is meant an amount that provides the desired treatment effect.

The polypeptides of the present invention in a pharmaceutical carrier can be administered to the subject, patient, or cell by injection (e.g., intravenous, intraperitoneal, subcutaneous, intramuscular), or by other methods such as infusion that ensure its delivery to the bloodstream in an effective form. The polypeptides may also be administered orally, intranasally, via aerosol delivery or via mucosal delivery.

Provided herein is a method of modulating a humoral immune response in a subject, comprising administering to the subject an antibody having the same epitope specificity as an antibody produced by the hybridoma cell line deposited with the ATCC as hybridoma 4-2A6, 1-5A3, or 1-3B4, or a fragment thereof. Optionally, the humoral immune response is enhanced. For example, the humoral immune response can be enhanced in a subject with an immunodeficiency, or the humoral immune response can be enhanced in a subject with an infectious disease. Optionally, the humoral immune response is reduced.

By “modulation” is meant either enhancing or reducing the humoral immune response. One of skill in the art would know how to select an antibody in order to activate or inhibit the FcRH1 or FcRH4 receptor to effect the desired modulation of the humoral immune response. Immune modulation may include co-ligation of FcRH1 or FcRH4 with another cell molecule (i.e. CD20 or another FcRH) to enhance or reduce the immune response. Thus, in the case of an allergic response, one skilled in the art would choose to reduce the humoral immune response. In the case of exposure of a subject to an infectious agent (e.g., a viral or bacterial agent), one skilled in the art would choose to enhance the humoral antibody response.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Cells

Human and mouse cell lines were cultured in RPMI1640 medium containing 100 U/ml penicillin, 100 mg/ml streptomycin, 2 mM L-glutamine, and 10% fetal calf serum (Life Technologies, Grand Island, N.Y.). Human blood samples, tonsils, and rib sections were obtained. Mononuclear cells in these tissues were isolated by Ficoll-Hypaque gradient centrifugation. Naïve B cells in tonsil samples were purified to >90% purity by depletion of CD10⁺, CD27⁺, CD38⁺, CD3⁺, and CD14⁺ cells using monoclonal antibodies, antibody conjugated microbeads or goat anti-mouse IgG conjugated microbeads (Miltenyi Biotec, Auburn, Calif.). Stained cells were analyzed on a FACSCalibur flow cytometer (BD Biosciences, Mountain View, Calif.) and plotted using WINMDI software (Scripps Institute, La Jolla, Calif.).

### Production of Monoclonal Anti-FcRH1 Antibodies

Balb/c mice hyperimmunized with baculovirus derived recombinant FcRH1 extracellular region protein (10 μg/injection) were boosted with Daudi, Ramos and Raji cells on the day before fusion of regional lymph node cells with the Ag8.653 plasmacytoma cell line.¹⁵ Hybridoma supernatants were screened by ELISA for anti-FcRH1 antibody activity before testing for immunofluorescence reactivity with B cell lines and Western blotting of recombinant FcRH1-5 proteins. Hybridomas producing anti-FcRH1 specific antibodies were subcloned by limiting dilution, and the antibody isotype determined by an indirect capture ELISA (Zymed, San Francisco, Calif.).

### Western Blot Analysis

Baculoviral derived recombinant FcRH1-5 ectodomains were made and Recombinant FcγR proteins were obtained. Recombinant FcRH and control proteins (1 μg each) were resolved by SDS-PAGE and transferred onto nitrocellulose membranes. Antibody reactivity was assessed by incubation of these protein loaded membranes with test antibodies (3 μg/ml) and horseradish peroxidase-labeled goat anti-mouse Ig antibody (1:5000 dilution; Dako, Denmark). Antigen-antibody reactivity was visualized by enhanced chemiluminescence (Amersham Life Science, Piscataway, N.J.). A HA-FcRH1 chimeric receptor overexpressing IIA1.6 cell line was established by known methods.¹⁷ Before and after FcRH1 ligation by anti-FcRH1 antibody, cell lysate (200 μg) was immunoprecipitated with anti-hemagglutinin (HA) antibody (Roche Diagnostics, Mannhein, Germany) and the immunoprecipitates were immunoblotted with either anti-phosphotyrosine antibody (4G10, Upstate Biotechnologies, Lake Placid, N.Y.) or anti-FcRH1 antibody.

### Immunofluorescence Cell Sorting and RT-PCR Analysis of FcRH1 Transcript Expression

Tonsillar B cell subpopulations were purified by immunofluorescent cell sorting with a MoFlow instrument (Cytomation, Fort Collins, Colo.) as follows: naïve cells (CD2T CD38⁻IgD⁺ CD19⁺), pre-GC cells (CD3 ⁺IgD⁺CD19⁺), centroblasts (CD77⁺ CD38⁺ CD19⁺), centrocytes (CD77⁻CD38⁺ CD19⁺), memory B cells (CD27⁺ CD38⁻CD19⁺), and plasma cells (CD38⁺⁺IgD⁻CD19⁺). Sorted cells were lysed in TRIzol reagent (Gibco, Grand Island, N.Y.) before preparation of total RNA and first-stand cDNA synthesis using Superscript II system (Invitrogen, Carlsbad, Calif.). After inactivating the reactions 50° C. for 2 min real-time PCR was performed by using SYBR Green PCR Master Mix (Applied Biosystems, Foster City, Calif.) denaturing at 95° C. for 10 min, amplification for 40 cycles at 95° C. for 15 sec, annealing and extension at 60° C. for 1 min using an ABI Prism 7900 HT sequence detection system (Applied Biosystems, Foster City, Calif.). FcRH1 gene specific primers used for PCR amplification were 5′-AGGAGATCCCAGATAAATGTG-3′ and 5′-CTGTGCCCATAGCAACTGAG-3′.

### Transient Transfectants

Full length FcRH1-5 cDNAs were ligated into the pEGFP-N1 mammalian expression vector (Clontech, Palo Alto, Calif.). 5 μg of purified plasmid was transfected into the human 293T cell line using Lipofectamine reagent (Invitrogen, Carlsbad, Calif.). Transfectants were harvested at 48 hours and stained for reactivity with FcRH1 antibodies. FcRH2-5 surface expression was confirmed by reactivity with FcRH specific antibodies.

### Antibodies, Immunofluorescence Reagents and Chemicals

Fluorescein isothiocyanate (FITC)-conjugated anti-human CD3, CD27, and CD34 antibodies, phycoerythrin (PE)-conjugated anti-human CD19, CD3, CD14, CD56, CD38, and IgD antibodies, and allophycoreythrin (APC)-conjugated anti-human CD34 and CD19 antibodies were purchased from Becton Dickinson (Mountain View, Calif.). Streptavidin-PE, streptavidin-APC, streptavidin, FITC-conjugated anti-human IgM, and FITC-conjugated anti-rat Ig antibodies were from Southern Biotechnology Associates (Birmingham, Ala.). Monoclonal anti-human CD77 antibody was from Coulter/Immunotech (Marseille Cedex, France). Immobilized pepsin and sulfo-NHS-LC-biotin were obtained from Pierce (Rockford, Ill.).

### B Cell Activation, Cell Cycle Status and Proliferation Assays

B cells purified from tonsils were incubated in 96-well plates (10⁵/well in 200 μl RPMI supplemented with 10% FCS) for 72 hr in the presence or absence of biotinylated Fab fragments of anti-FcRH1 mAbs with 20 μg/ml streptavidin. Cells pulsed for an additional 16 hr with ³H-thymidine (1 μCi/well) were then harvested and ³H-thymidine incorporation was assessed with a liquid scintillation counter. Cell surface expression of IgD, IgM, CD69, CD80, and CD86 assessed before and after naïve B cells (5×10⁵/24-well) were incubated for 48 hr in the presence or absence of biotinylated Fab fragments of anti-FcRH1 mAbs with 20 μg/ml streptavidin. To evaluate the cell cycle status, sorted cells were fixed with 100% ethanol, treated with RNAase A, and stained with 40 μg/ml propidium iodide. DNA content was assessed using a FACSCaliber flow cytometer (Becton Dickison, Mountain View, Calif.).

### Calcium Mobilization and Apoptosis Assays

Cells (5×10⁶) were washed twice with Hank's balanced salt solution (HBSS) before re-suspension in 1 ml HBSS containing the indication dye, Fluo-4-AM (2 μM), and reference dye, SNARF-1 (4 μM) (Molecular Probes, Eugene, Oreg.). After incubation for 30 min at 37° C., cells were washed twice with HBSS, and the Ca²⁺ levels were measured before and after receptor ligation using a FACSCaliber flow cytometer. Calcium concentrations detected by Fluo-4 AM were normalized to the readouts by SNARF-1 and illustrated as the Fluo-4/SNARF-1 ratio. Apoptotic cells were identified with an In Situ Cell Detection kit using terminal deoxynucleotidyl transferase-mediated dUTP nick end labeling (TUNEL) (Roche Diagnostics, Indianapolis, Ind.). Cells fixed with 2% paraformaldehyde for 15 min were permeablized with 0.1% Triton-100 in 0.1% sodium citrate for 3 min and incubated with deoxynucleotidyl transferase in labeling buffer for 1 hr at 37° C. The percentage of TUNEL positive cells was determined by flow cytometric analysis.

### Statistics Analysis

The Student's t test was used to evaluate the significance of differences in experimental results.

### Generation of Monoclonal Anti-FcRH1 Antibodies

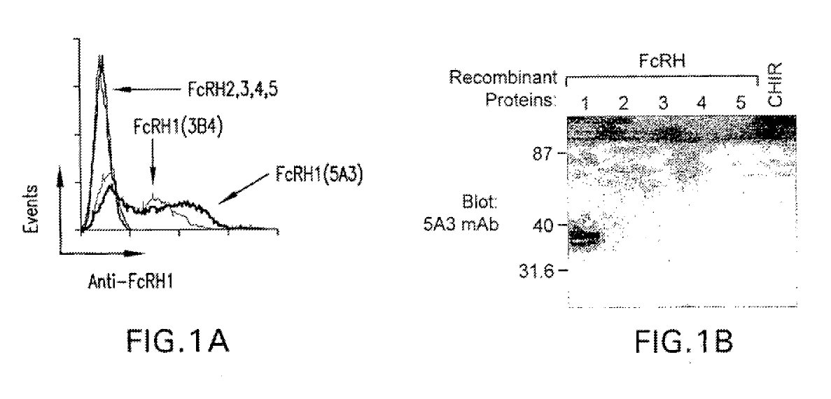

Lymph node cells from mice hyperimmunized with recombinant protein corresponding to the three extracellular Ig domains of FcRH1 and boosted with FcRH1 transcript positive human B cell lines (Davis, R. S. (2001) Proc. Natl. Acad. Sci. 98, 9722-9777) were fused with a non-Ig producing plasmcytoma cell line. Antibodies produced by 12 hybridoma clones were found to be reactive with an FcRH1-transfected cell line when assessed by cell surface immunofluorescence. The fine specificity of two of these monoclonal antibodies, 5A3 (γ_{2b}κ) and 3B4 (γ₁κ), were shown by their lack of reactivity with other FcRH family members by immunofluorescence analysis of transfected cells (FIG. 1A). The specificity was confirmed by Western blot assays demonstrating that the 5A3 antibody reacted with the recombinant FcRH1 protein, but not with other FcRH family members (FcRH2-5), chicken Ig-like receptor (CHIR), FcγRIIa, FcγRIIb, and FcγRIII (FIG. 1B). Likewise, the 3B4 antibody was found to be specific for FcRH1, except that it did not react with recombinant FcRH1 protein in Western blots. This data demonstrates that both the 5A3 and 3B4 mAbs specifically recognize native FcRH1 molecules. Biotinylated Fab monomers of these antibodies were prepared for use in identification and ligation of the FcRH1 molecules on B lineage cells.

### FcRH1 is Variably Expressed as a Function of B Cell Differentiation

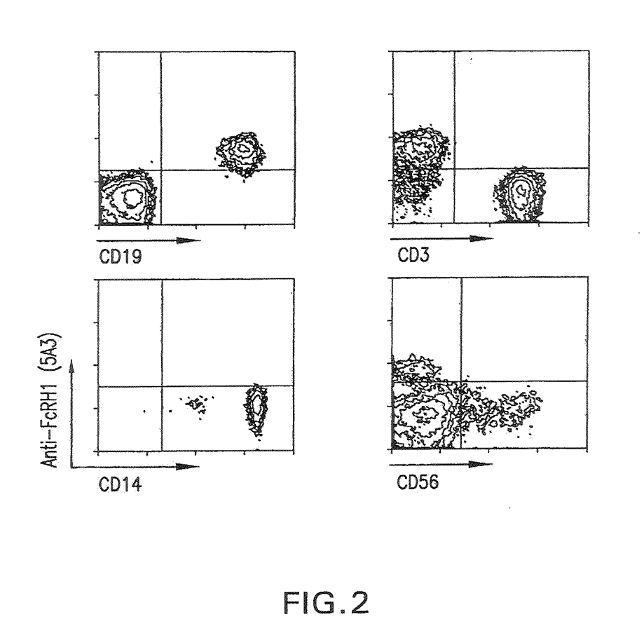

Previous analysis of FcRH1 transcripts in blood and tonsillar cells has suggested that FcRH1 expression is B lineage specific (Davis, R. S. (2001) Proc. Natl. Acad. Sci. 98, 9722-9777; Miller, I (2002) Blood 99, 2662-2669). To determine which cells express the FcRH1 protein, blood mononuclear cells were stained with biotinylated Fab fragments of the 5A3 anti-FcRH1 mAb and streptavidin-APC in conjunction with PE-labeled mAbs against lineage specific markers. FcRH1 was found on all of the circulating B cells, and not on T cells, NK cells, monocyte/macrophages, granulocytes and platelets (FIG. 2). RT-PCR analysis of FcRH1 transcripts in by B cells, T cells, NK cells, and monocyte/macrophages confirmed the exclusive expression of FcRH1 by B cells. FcRH1 protein expression was correspondingly found on human B cell lines and not on T, monocytoid, or erythroid cell lines (Table 2).

TABLE 2
Expression of FcRH1 on cell lines

| Cell type | Cell line | Cell surface staining by anti-FcRH1 |
|---|---|---|
| Pro-B | Nalm16 | − |
| Pre-B | 697 | − |
| | 207 | − |
| B | Daudi | +++ |
| | Raji | + |
| | Ramos | +++ |
| | BJAB | + |
| T | Jurkat | − |
| Monocytic | THP-1 | − |
| Myelomonocytic | U937 | − |
| Erythroid | K562 | − |

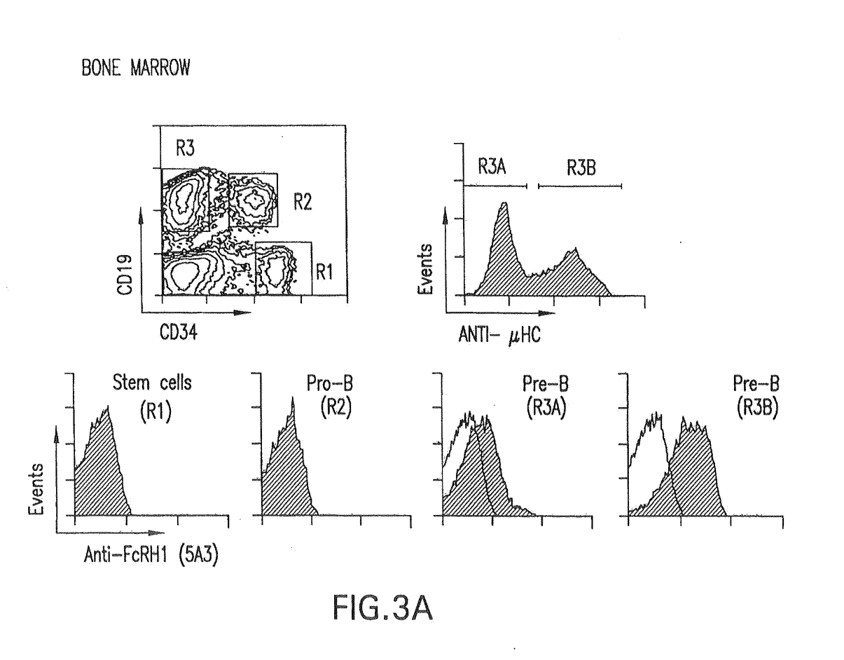

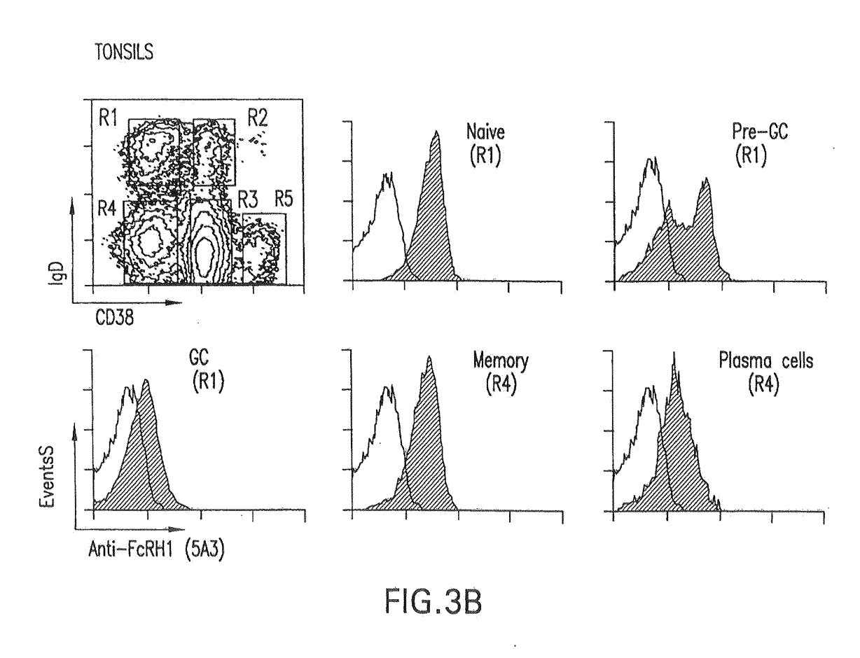

Notably, FcRH1 was also undetectable on pro/pre-B cell lines and, correspondingly, was found on only a subpopulation of the CD19⁺ B lineage cells in the bone marrow. The hematopoietic stem cells (CD34⁺ CD19⁻) and pro-B cells (CD34⁺ CD19⁺) did not express detectable FcRH1 (FIG. 3A, R1 and R2), while most cells (60-89%) in a mixed pre-B and B cell population (CD34⁻CD19⁺) expressed FcRH1. When this population was divided into pre-B and immature B cell subpopulations according to their μ heavy chain expression levels (R3A and R3B subpopulations in FIG. 3A), relatively low levels of FcRH1 expression were found on pre-B cells and higher levels were found on B cells.

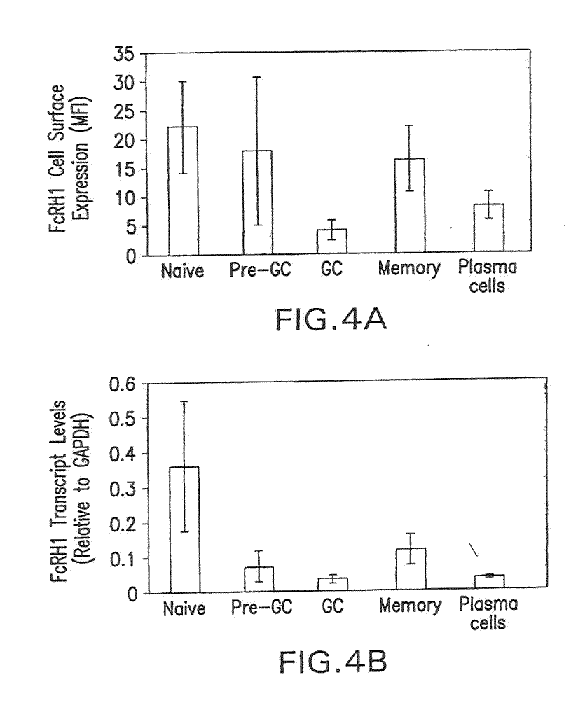

To examine FcRH1 expression by B lineage cells in secondary lymphoid tissues, the CD19⁺ B cells isolated from tonsil samples were subdivided on the basis of their differential expression of cell surface IgD and CD38 (Pascual, V (1994) J. Exp. Med. 180, 329-339). into follicular mantle (IgD⁺CD38⁻), pre-germinal center (pre-GC, IgD⁺CD38⁺), germinal center (GC, IgD⁻CD38⁺), memory (IgD⁻CD38⁻), and plasma cells (CD38⁺⁺). Relatively high levels of cell surface FcRH1 expression were found on the naïve (follicular mantle) B cells (FIG. 3B, R1). Whereas GC B cells and plasma cells (FIG. 3B, R3 and R5) expressed FcRH1 at very low levels, memory B cells were found to express FcRH 1 in levels almost as high as those found on naïve B cells (FIG. 3B, R4). For the tonsillar pre-GC population the levels of FcRH1 expression were remarkably variable in different donors, presumably reflecting differences in their antigenic activation status. In 2 of 7 tonsils, FcRH1 surface expression levels divided the pre-GC cells into relatively high- and low-expressing subpopulations (See FIG. 3B, R2). The pre-GC cells in two donor samples expressed higher FcRH1 levels, while those in the three remaining samples had medium to low FcRH1 levels. When FcRH1 transcript levels were examined by real-time RT-PCR analysis of the different subpopulations of purified tonsillar B cells, the naïve B cells were found to express the highest levels of FcRH1 transcripts, while the pre-GC and memory B cells expressed intermediate levels. The lowest B cell levels of FcRH1 transcripts were found for the GC B cells and plasma cells (FIG. 4A). This analysis demonstrates that FcRHJ transcription begins in pre-B cells, increases as B cells mature, is down-regulated as B cells are activated to form germinal centers and later to undergo plasma cell differentiation. Memory B cells regain FcRH1 expression, although at lower levels than for naïve B cells.

### FcRH1 Down-Regulation Occurs During Pre-Germinal Center Activation

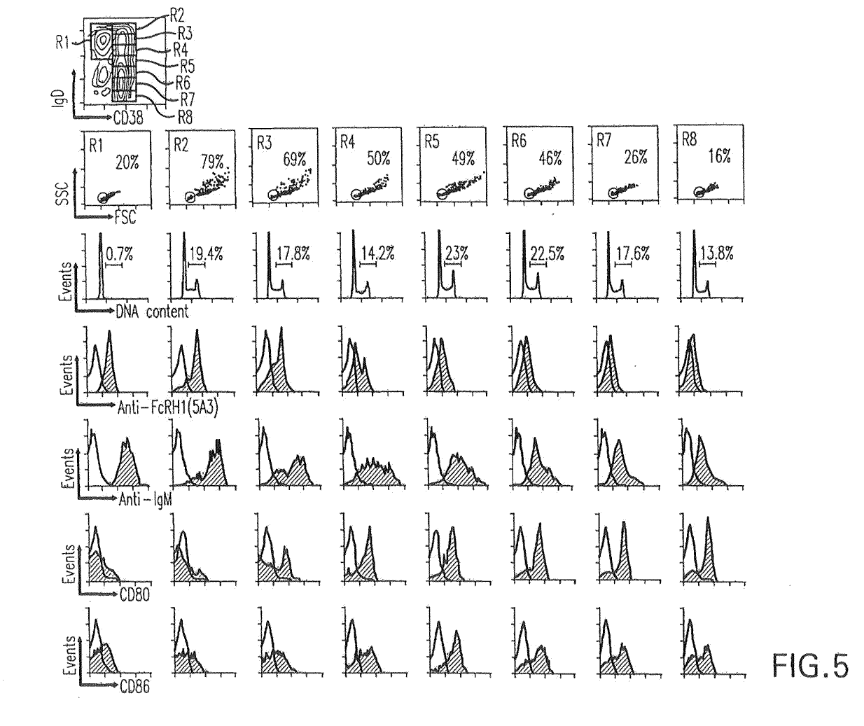

To examine more precisely the FcRH1 down-regulation that occurs during the naïve to GC B cell transition, the naïve, pre-GC, and GC cells were divided into multiple subsets on the basis of their cell surface IgD and CD38 levels (FIG. 5). When these subsets were examined for expression of FcRH1 and other cell surface molecules, FcRH1 levels were found to be uniformly high on naïve B cell subsets. In contrast, coinciding with the onset of CD38 expression by pre-GC cells, progressive FcRH1 down-regulation occurred in concert with diminishing levels of cell surface IgD and IgM, and this pattern was maintained for GC B cells (FIG. 5). A dramatic increase in cell size was also observed with the onset of CD38 expression, presumably reflecting activation of the B cells entering the pre-GC compartment. The size of pre-GC cells then progressively decreased in concert with the decline in their IgD and IgM expression levels (FIG. 5). Cell cycle entry also coincided with the initiation of CD38 expression. Whereas >99% of the naïve B cells were in the Gₒ or G₁ phases, 14-23% of B cells were found to be in the S and G₂/M phases throughout the pre-GC and GC stages (FIG. 5). Expression of the CD80 and CD86 costimulatory molecules was also up-regulated after the B cells entered the pre-GC stage (FIG. 5). This constellation of findings demonstrates that FcRH1 is well positioned to potentially influence the activation of naïve B cells.

### B Cells are Activated by FcRH1 Ligation

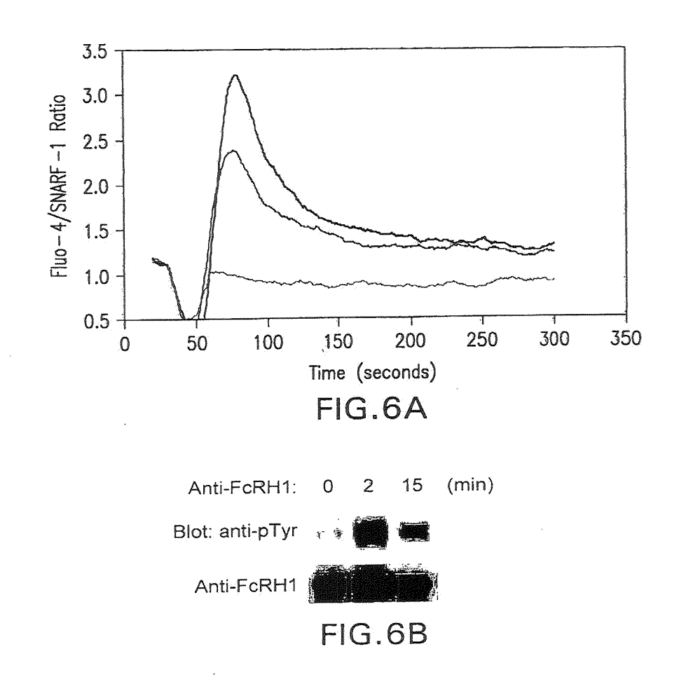

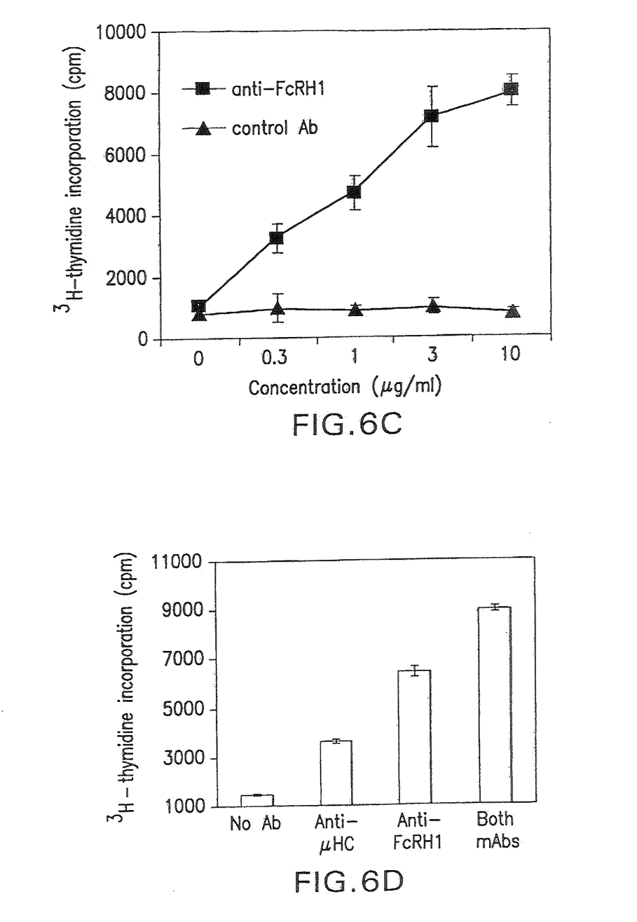

To examine the activation potential for FcRH1, B cells of the Daudi cell line were treated with biotinylated anti-FcRH1 Fab fragments plus streptavidin. FcRH1 ligation alone had no demonstrable effect on intracellular calcium levels, whereas concomitant FcRH1 ligation enhanced the Ca²⁺ flux induced by BCR ligation (FIG. 6A). Also consistent with its possession of ITAM-like motifs, transient FcRH1 tyrosine phosphorylation was observed after its ligation on an FcRH1 transfected cell line (FIG. 6B). When the FcRH1 activation potential was examined for native B cells in tonsillar samples, FcRH1 ligation was found to induce a significant increase in the proportion of relatively large B cells, 38.2±1.0 vs. 24.7±1.3 for unstimulated control cells (p=0.01), and of CD69⁺ cells (59.0±0.9 vs. 33.4±1.9 for controls, p=0.037) after 48 hrs in culture. Conversely, surface IgD levels were reduced by FcRH1 cross-linkage (mean fluorescent intensity of 25.9±0.1 for control cells vs. 18.8±0.5 for stimulated cells, p=0.04), whereas the proportion of CD86⁺ cells was enhanced, 40.0±1.1 vs. 15.6±0.4 for unstimulated cells (p=0.02). The ligation of FcRH1 on tonsillar B cells also induced an increase in ³H-thymidine uptake (FIG. 6C), and FcRH1 and BCR co-ligation resulted in an additive effect on B cell proliferation when suboptimal doses of anti-IgM were used (FIG. 6D). The ability of FcRH1 to induce B cell proliferation was confirmed by the finding of an anti-FcRH1 dose-related increase in cell numbers without an accompanying alteration of cell survival (42.6±2.0 TUNEL-positive cells vs. 43.7±1.9 for unstimulated control cells at 24 hr, p=0.898). FcRH1 ligation also had no effect on the expression levels of anti-apoptotic proteins (Bcl-2, Bcl-xL, and Mcl-1) or a pro-apoptotic protein (Bax).

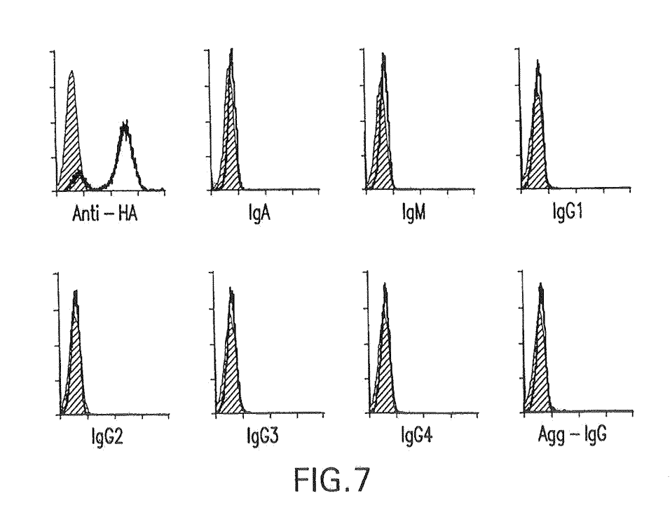

Whether FcRH1 was an Fc receptor was examined, because of the close relationship between FcR and FcRH gene families, by incubating FcRH1-transfected cell lines with either soluble or aggregated forms of human IgM, IgA, IgG₁, IgG₂, IgG₃, and IgG₄ and subsequent immunofluorescence assessment of binding. The results of these analyses did not reveal evidence of FcRH1 binding for any of these human Ig isotypes (FIG. 7) These results indicate that FcRH1 does not serve as an Fc receptor for IgM, IgG, or IgA antibodies.

### Example 2

### Antibodies and Reagents

Antibodies to FcRH4 were generated and coupled to biotin (Pierce Biotechnology, Rockford, Ill.) or directly labeled with Alexa647 (Molecular Probes, Eugene, Oreg.). F(ab′)₂ fragments of the FcRH4 monoclonal antibodies were generated by pepsin digest (Pierce Biotechnology, Rockford, Ill.) according to the manufacturer's instructions. Biotinylated anti-IgD and Streptavidin-PE were purchased from Southern Biotech Associates (Birmingham, Ala.). All other antibodies and reagents for FACS analysis were obtained from BD-Pharmingen (San Diego, Calif.). Polyclonal mouse anti-human Ig was obtained from Jackson Immunoresearch and SAC was obtained from Sigma. Recombinant IL-2, IL-4 IL-10 and CD40L were purchased from R&D Systems (Minneapolis, Minn.). Plates for Elispot assays were purchased from Millipore (Billerica, Mass.) and the substrate for the HRP-labeled anti-human Ig for Elispot assays were obtained from Moss Inc (Pasadena, Md.).

### Generation of Anti-FcRH4 Monoclonal Antibodies

Monoclonal antibodies to FcRH4 were generated by fusing B cells from mice immunized with Baculovirus recombinant protein of the extracellular region of FcRH4 to Ag8 cells. Approximately 200 clones were analyzed by ELISA on 96-well plates coated with recombinant extracellular domain of FcRH4. Clones that were positive by ELISA were further analyzed for specificity by FACS analysis using A20-IIA1.6 cells stably expressing HA-epitope tagged FcRH4 and by immunoprecipitation experiments. Western blotting and immunoprecipitation experiments were performed by known methods (Ehrhardt, G. R. et al. Proc Natl Acad Sci U S A 100, 13489-94 (2003)).

### Cells

All cells were maintained in RPMI supplemented with 10% FCS, 2 mM L-glutamine, 100 units/ml penicillin/streptomycin and 50 f3-mercapto-ethanol. Tonsils were obtained from the Childrens Hospital, University of Alabama at Birmingham, Birmingham, USA. Single cell suspensions were obtained by mincing of the tonsils through a 70 mm mesh followed by centrifugation over a ficoll-hypaque gradient. For FACS analysis the cells were purified with anti-CD 19 coupled to magnetic beads (Miltenyi Biotechnologies, Burgisch Glasbach, DE) followed by magnetic separation to obtain a >99% pure B cell population. The cells were labeled with the indicated antibody combinations and analyzed using a FACS-Calibur instrument (BD-Pharmingen, San Diego, Calif.). To assess the morphology of FcRH4 positive and negative populations, respectively, the cells were spun down onto glass slides and stained with a Giemsa-Wright stain (Sigma, St. Louis, Mo.). For Ig secretion assays, tonsillar B cells were obtained by depleting non-B cells using the B cell Isolation Kit II kit (Miltenyi, Burgisch Gladbach, DE). A >99% pure B cell population was then stained with anti-IgD, anti-CD38 and anti-FcRH4 antibodies and FcRH4-positive and FcRH4-negative cells were purified using a MoFlow FACS sorter.

### Sequence Analysis of V_{H} Genes

First strand cDNA was generated by performing random primed RT-PCR on CD 19-positive tonsillar B cells that were purified by FACS-sorting into IgD⁻/CD38⁻/FcRH4⁻ and IgD⁻/CD38⁻/FcRH4⁺ subpopulations. For the first round of PCR amplification with a high fidelity PCR-polymerase (Invitrogen, Carlsbad, Calif.), a primer mix recognizing all V_{H}-gene family members and J_{H}-gene family members was used (for primer sequences see Küppers, R., Hansmann, M. L. & Rajewsky, K. (1997) in Weir's Handbook of Experimental Immunology, eds. Herzenberg, L. A., Weir, D. M. & Blackwell, D. (Blackwell Scientific, Oxford), pp. 206.1-206.4). For the second round of PCR-amplification, only V_{H}3-gene specific primers were used in conjunction with primers recognizing all three J_{H}-genes. The resulting PCR products were cloned into pBlueScript for sequence analysis. Analyzed were 264 nucleotides encompassing the framework 1, 2 and 3 regions as well as the CDR1 and CDR2 regions of the indicated V_{H} genes. Sequence analysis was performed using the IMGT, the international ImMunoGeneTics Information System® (http://imgt.cines.fr) (Lefranc, M. P. Nucleic Acids Res 31, 307-10 (2003)).

### ELISA

ELISA plates were coated with a mixture of mouse anti-human IgA, IgM and IgG (2 mg/ml). For Ig secretion analysis 15,000 FcRH4-positive or FcRH4-negative memory B cells per well were plated in a volume of 150 ml and incubated with the indicated factors (IL-2 60 ng/ml, IL-10 200 ng/ml, CD40L 2 mg/ml, SAC 0.001%) for 4 days before the supernatants were added to the coated ELISA plates. After over night incubation at 4° C., the plates were washed and a secondary HRP-labeled goat anti-human Ig antibody was added for 1 hour at room temperature. Following incubation with the secondary antibody, the plates were washed again and HRP-substrate was added for 30 min. The plates were read using a microplate reader at 405 nm.

### Quantitative RT-PCR Analysis

For quantitative RT-PCR analysis, mRNA from sorted populations was generated using the RNeasy Kit (Qiagen, Valencia, Calif.). Random primed cDNA corresponding to 5000 cells/reaction was used as template. Oligos were designed to overlap exon-intron borders to avoid potential amplification of contaminating genomic DNA. Quantitative RT-PCR was performed using SYBR-Green PCR Master Mix (Applied Biosystems, Foster City, Calif.) on a 7900HT Sequence Detection System (Applied Biosystems, Foster City, Calif.).

### Cell Proliferation Assays

FcRH4-positive or FcRH4-negative memory B cells were plated at a density of 15,000 cells per well in round bottom 96-well plates. The cells were treated for 40 hours with the indicated stimuli (intact anti-Ig 2 mg/ml, F(ab′)₂ anti-Ig 1.33 mg/ml, cytokines and SAC as described above) before addition of 1mCi ³H-thymidine for an additional 10 hours. The cells were then harvested using a Basic96 Harvester (Skatron Instruments, Norway) and thymidine incorporation was measured using a Wallac liquid scintillation counter.

### Generation of Anti-FcRH4 Specific Antibodies

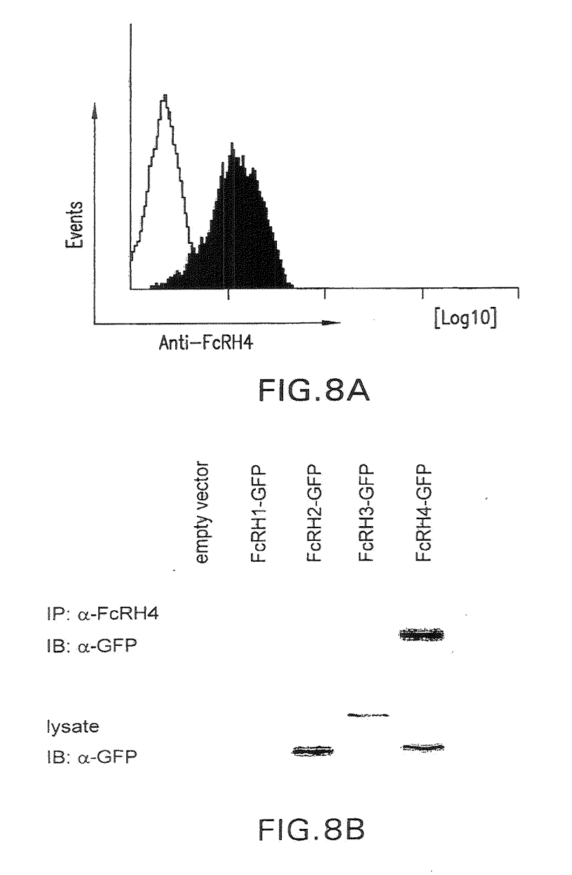

Monoclonal antibodies were generated to FcRH4 by immunizing mice with recombinant protein encompassing the extracellular domain of FcRH4. To verify specificity of one promising anti-FcRH4 monoclonal antibody, A20-IIA1.6 cells were transiently transfected with a construct in which GFP was fused c-terminally to FcRH4 and stained with the anti-FcRH4 antibody. A clear shift could be observed on GFP-positive cells. In contrast, no signal was obtained in vector control transfected cells (FIG. 8A). Whether this antibody was specific in immunoprecipitation experiments was then tested. For this purpose lysates from BOSC23 cells transiently transfected to express various FcRH-GFP-fusion proteins or vector control cells were subjected to immunoprecipitation using anti-FcRH4 antibodies. As shown in FIG. 8B, the anti-FcRH4 antibodies specifically immunoprecipitated FcRH4-GFP fusion proteins, but none of the other transfected FcRH-family members fused to GFP.

### FcRH4 Protein Expression is Restricted Mainly to a Subset of IgD⁻/CD38⁻ Memory B Cells

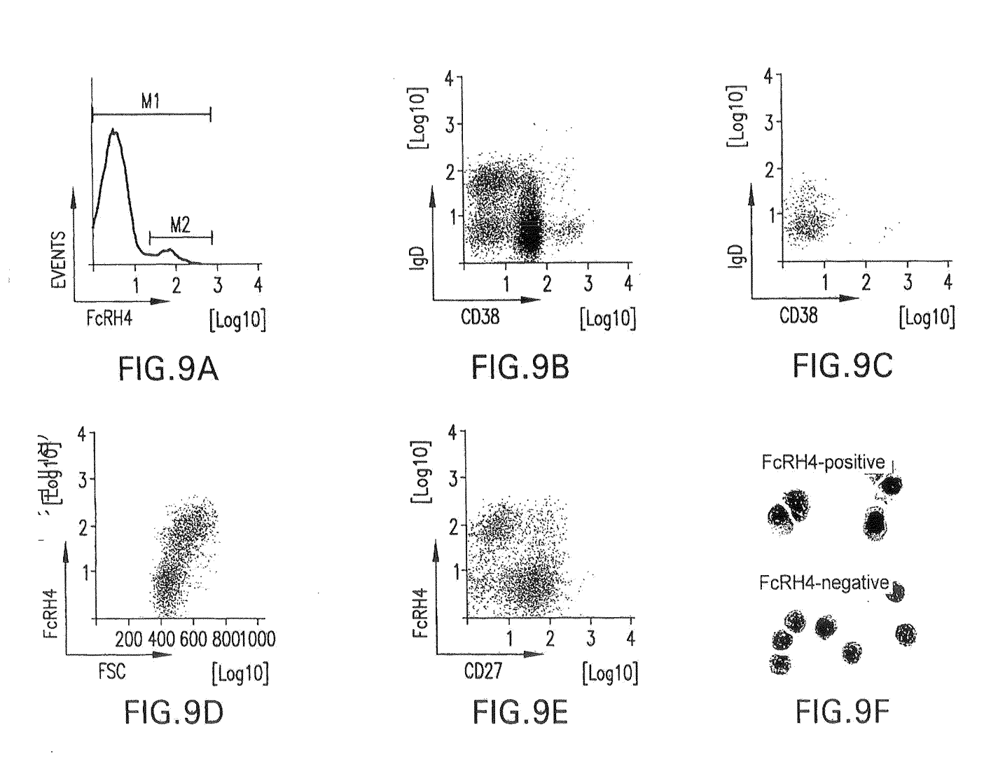

It has been demonstrated that FcRH4 mRNA was confined to memory B cells in the tonsil (Ehrhardt, G. R. et al. Proc Natl Acad Sci U S A 100, 13489-94 (2003)). To investigate the pattern of expression of FcRH4 protein CD19-positive human tonsillar B cells were analyzed. Staining of these tonsillar B cells showed a subpopulation of 9.49% (+1-4.95 SD, n=24) that were positive for FcRH4 expression (FIG. 9A). Earlier work (Bohnhorst, J. O., et al., J Immunol 167, 3610-8 (2001); Pascual, V. et al. J Exp Med 180, 329-39 (1994)) demonstrated that IgD and CD38 are useful markers to divide tonsillar B cells into naïve (IgD⁺/CD38⁺), pre GC (IgD⁻/CD38⁺), GC (IgD⁻/CD38⁺), plasma (IgD−/CD38⁺⁺) and memory cell subpopulations (IgD⁻/CD38⁻). Co-staining of CD19⁺ MACS-purified tonsillar B cells with anti-IgD, anti-CD38 and anti-FcRH4 indeed resulted in those clearly defined subpopulations (FIG. 9B). As expected from quantitative mRNA analysis (Ehrhardt, G. R. et al. Proc Natl Acad Sci U S A 100, 13489-94 (2003)), the vast majority of FcRH4 positive cells were found in the IgD7CD38″ memory B cell fraction (FIG. 9C). On average, approximately one third (34.98%+/−11.16% SD, n=11) of IgD⁻/CD38⁻ cells were positive for FcRH4 expression. FcRH4-positive cells had an increased cell size compared to the FcRH4-negative cells from the IgD⁻/CD38⁻ memory gate (FIG. 9D). Importantly, co-staining with CD27, a cell surface protein commonly used as memory B cell marker, revealed that FcRH4-positive cells were mostly CD27-negative whereas the FcRH4-negative cells from the memory gate were overwhelmingly CD27-positive (FIG. 9E). GIEMSA-staining of purified FcRH4-positive and FcRH4-negative IgD⁻/CD38⁻ memory B cells revealed a distinctive morphology of FcRH4-positive cells that underscored their increased size and with more cytoplasm (FIG. 9F).

### FcRH4-Positive Cells Display an Activated Phenotype

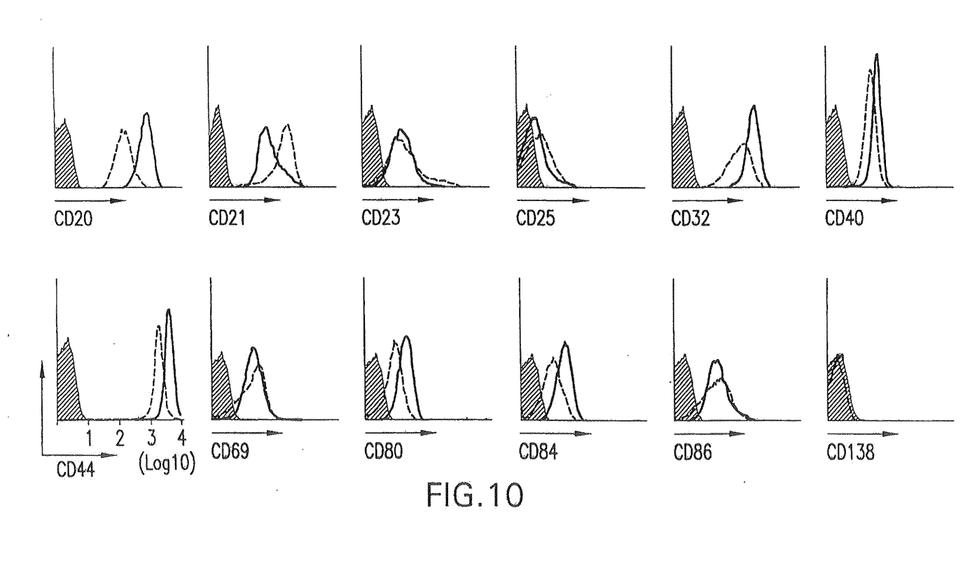

As the vast majority of FcRH4-positive cells were found in the IgD⁻/CD38⁻ memory B cell gate, these cells were analyzed for co-expression of cell surface markers commonly associated with memory B cells. FcRH4-positive cells, just like their FcRH4-negative counterparts were positive for CD20, CD21, CD23, CD32, CD40, CD44, CD69, CD80, CD84 and CD86 but negative for the plasma cell marker CD138 (FIG. 10). Expression of the alpha chain of the IL2 receptor was low to undetectable. With the exception CD20, which was higher expressed on FcRH4-positive cells than on FcRH4-negative cells. Most of the analyzed markers indicated slightly higher levels of expression on FcRH4-positive cells then on FcRH4-negative cells. However, FACS-analysis measures total protein expression on a given cell but does not account for cell size. Thus, the moderately higher expression levels on FcRH4-positive cells should not translate into a higher receptor density per cell. In contrast, the larger FcRH4-positive cells expressed only low levels of CD21, also known as complement receptor 2 (CR2). This should translate into a further augmented difference in receptor density.

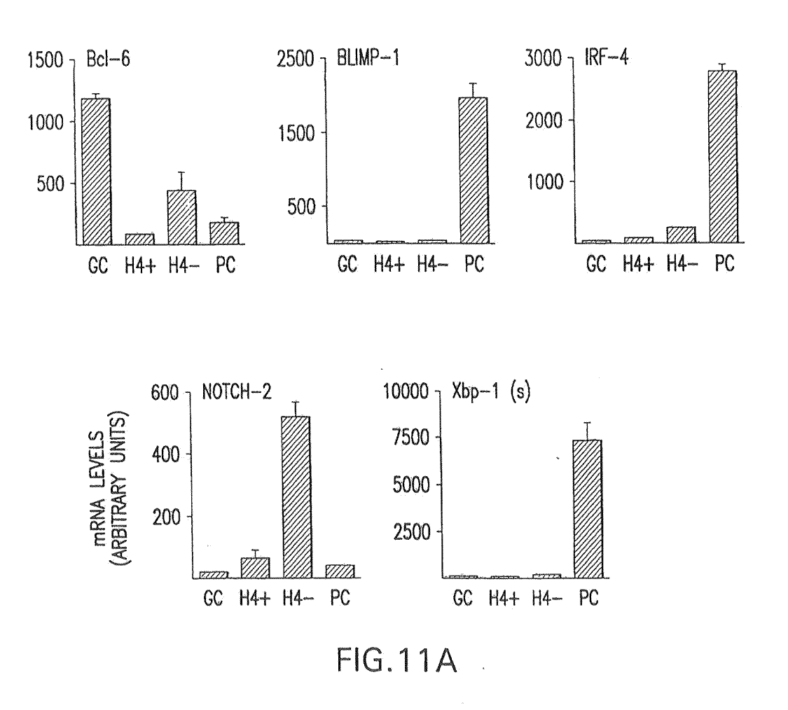

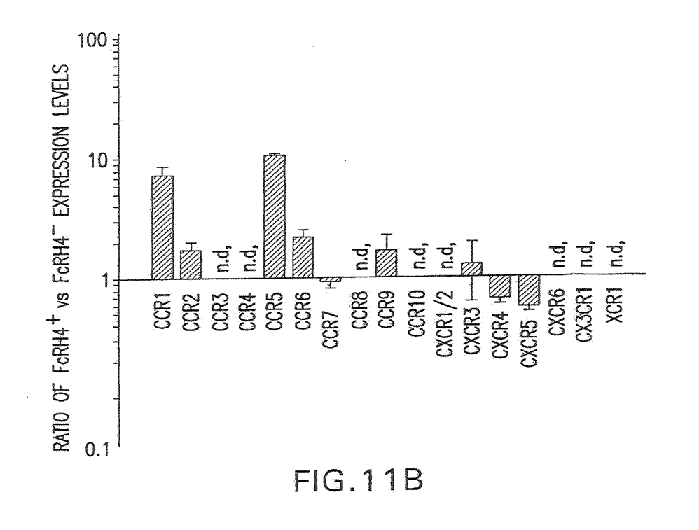

Although CD138 is commonly used as a plasma cell marker, data have been presented that not all plasma cells express CD138 (Ellyard, J. I. et al., Blood 103, 3805-12 (2004)). Therefore, mRNA levels of transcription factors that have been reported to drive plasma cell differentiation were analyzed, namely Blimp-1 (Shapiro-Shelef, M. et al., Immunity 19, 607-20 (2003)), the spliced isoform of Xbp-1 (Iwakoshi, N. N. et al., Nat Immunol 4, 321-9 (2003)) and IRF4 (Mittrucker, H. W. et al., Science 275, 540-3 (1997)), as well as Bcl-6 and Notch-2 in FcRH4-positive and -negative memory cells, germinal center cells and plasma cells. Transcripts of Blimp-1, Xbp-1 and IRF4 were barely detectable in FcRH4-positive and -negative memory B cells as well as in germinal center cells, as opposed to plasma cells were a very prominent signal was observed (FIG. 11A). Bcl-6 mRNA was detected in germinal center cells and, in reduced levels, in FcRH4-negative memory cells. Notch-2 mRNA could be amplified predominantly from FcRH4-negative memory B cells (FIG. 11A). Taken together, FcRH4-positive cells display features which demonstrate that they represent a specific subset of human memory B cells.

### Tissue Distribution and Chemokine Receptor Profile of FcRH4⁺ Versus FcRH4⁻ Memory B Cells

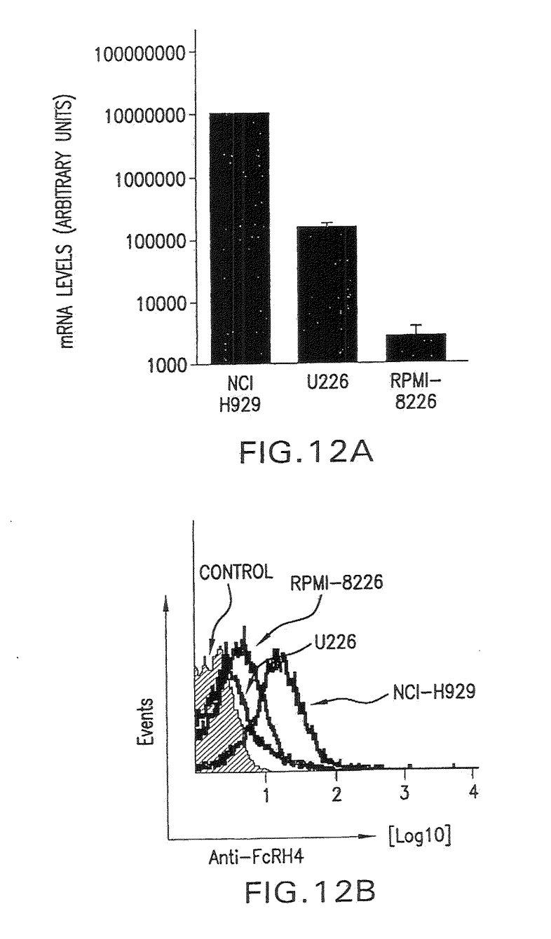

FcRH4 mRNA analysis and the demonstration that FcRH4 is expressed on a subpopulation of the memory B cells suggest stringent regulatory control of FcRH4 expression. Although cell surface FcRH4 is detectable on ˜10% of the tonsillar B cells, FcRH4⁺ B cells were rarely detected in bone marrow, spleen, and blood samples from healthy individuals. In contrast, CD27⁺ B cells were relatively abundant among B cell populations in the tonsils (53.0±12.7% SEM, n=11), blood (30.8±16.2% SEM, n=12), and spleen as expected. This highly selective pattern of FcRH4 expression was also reflected by the fact that almost all of the B lineage cell lines that were analyzed were negative for FcRH4 expression. The notable exceptions were multiple myeloma cell lines, three of which were found to express variable levels of FcRH4 mRNA and protein (Table 3 and FIG. 12)

Immunofluorescence analysis of FcRH4 expression by
CD19⁺ B cells in different tissues and B lineage cell lines

| | | FcRH4⁺ B cells |
|---|---|---|
| | | (no. of samples) |
| Tissue/cell lines | Tumor cell types | % |
| Bone marrow | | <0.5 (n = 2) |
| Peripheral blood | | <0.2 (n = 8) |
| Tonsils | | 9.5 ± 4.95 SEM |
| | | (n = 24) |
| Spleen | | <0.5 (n = 4) |
| Nalm16 | Acute lymphoblastic leukemia | <0.1 |
| | (pro-B cell line) | |
| 697 | Acute lymphoblastic leukemia | <0.1 |
| | (pre-B cell line) | |
| EU12 | Acute lymphoblastic leukemia | <0.1 |
| | (pro-B/pre-B cell line) | |
| Daudi | Burkitt's lymphoma (EBV⁺) | <0.1 |
| Raji | Burkitt's lymphoma (EBV⁺) | <0.1 |
| Namalwa | Burkitt's lymphoma (EBV⁺) | <0.1 |
| BJAB | Burkitt's lymphoma (EBV⁻) | <0.1 |
| Ramos | Burkitt's lymphoma (EBV⁻) | <0.1 |
| WSU-1 | Diffuse large B cell lymphoma | <0.1 |
| SUDHL-6 | Diffuse large B cell lymphoma | <0.1 |
| NCI-H929 | Multiple myeloma | 84 |
| RPMI-8226 | Multiple myeloma | 24 |
| U226 | Multiple myeloma | 10 |

CCR7 expression has been used to distinguish memory T cells (CCR7⁺) from effector T cells (CCRT), and the chemokine receptors expressed by these T cell subpopulations may influence their tissue localization pereferences. The chemokine receptor expression profiles for the FcRH4⁺ and FcRH4⁻ subpopulations of memory B cells was therefore surveyed. While differences were not seen for most chemokine receptors, including CCR7 and CXCR4, mRNA levels for CCR1 and CCR5 were strongly up-regulated in FcRH4⁺ cells in comparison with the FcRH4⁻ memory B cells (FIG. 11B). These findings suggest that localized production of the chemokine ligands for these two chemokine receptors may influence the tissue localization pattern of FcRH4⁺ memory B cells.

FcRH4-Positive Cells have Somatically Mutated V_{H} Regions

The hallmark characteristic of memory B cells is the appearance of somatic hypermutations in the variable regions of their rearranged immunoglobulin genes. To analyze the status of the V_{H} regions of FcRH4-positive and -negative cells V_{H}3-gene family regions were amplified by RT-PCR from FACS-purified cells.

TABLE 4

| | |
|---|---|
| FcRH4-negative | FcRH4-positive |

| | number of | | number of |
|---|---|---|---|
| V_{H}3-gene | mutations | V_{H} 3-gene | mutations |
| 3-20 | 0 | 3-7 | 1 |
| 3-9 | 2 | 3-33 | 4 |
| 3-7 | 3 | 3-23 | 4 |
| 3-23 | 5 | 3-23 | 6 |
| 3-48 | 5 | 3-7 | 7 |
| 3-74 | 5 | 3-23 | 7 |
| 3-9 | 6 | 3-7 | 8 |
| 3-7 | 7 | 3-15 | 8 |
| 3-66 | 7 | 3-23 | 8 |
| 3-48 | 7 | 3-30 | 9 |
| 3-7 | 9 | 3-23 | 9 |
| 3-23 | 10 | 3-64 | 10 |
| 3-9 | 13 | 3-30 | 10 |
| 3-23 | 14 | 3-23 | 11 |
| 3-11 | 14 | 3-30 | 12 |
| 3-15 | 14 | 3-23 | 13 |
| 3-23 | 15 | 3-15 | 13 |
| 3-30 | 15 | 3-30 | 13 |
| 3-23 | 15 | 3-64 | 14 |
| 3-11 | 16 | 3-9 | 14 |
| 3-74 | 17 | 3-23 | 16 |
| 3-23 | 18 | 3-23 | 16 |
| 3-30 | 18 | 3-23 | 16 |
| 3-11 | 19 | 3-23 | 17 |
| 3-11 | 44 | 3-48 | 20 |
| | | 3-53 | 34 |

Table 4 shows somatic hypermutation of FcRH4-positive and FcRH4-negative memory B cells. PCR-amplified cDNAs encoding re-arranged VH3-genes were cloned and sequenced. Analyzed sequences encompass the FR1,2 and 3, CDR1 and CDR2 regions. Rate of mutations detected in FcRH4-positive memory B cells was 11.54+/−6.44 (SD, n=26) and in FcRH4-negative memory B cells 11.92+/−8.71 (SD, n=25).

Sequence analysis revealed that both, FcRH4-positive and FcRH4-negative V_{H}-regions displayed a comparable frequency of somatic hypermutations. Therefore, FcRH4-positive cells from the IgD−/CD38− “memory” gate are in fact memory B cells.

### FcRH4-Positive and FcRH4-Negative Cells Display Distinct Proliferation and Differentiation Characteristics Upon Stimulation

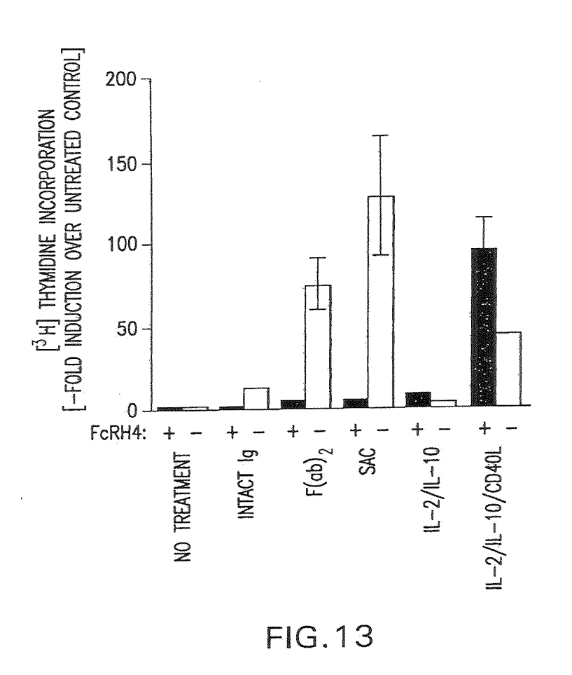

Ligation of the BCR has been demonstrated to induce a proliferation of memory B cells (Galibert, L. et al., J Exp Med 183, 2075-85 (1996)). Previously, it has been shown that the intracellular domain of FcRH4 is a very potent inhibitor of BCR-signaling, using the mouse memory B cell line A20-IIA 1.6 as a model system (Ehrhardt, G. R. et al., Proc Natl Acad Sci U S A 100, 13489-94 (2003)). To investigate the growth characteristics of FcRH4-positive and FcRH4-negative human tonsillar memory B cells, FACS-purified memory B cells were stimulated for 40 hours by addition of anti-Ig antibodies (intact antibodies or F(ab′)₂-fragments). In addition the cells were also stimulated by addition of the polyclonal activator SAC or the cytokines IL-2/IL-10 and CD40L. Analysis of thymidine incorporation assays revealed that FcRH4-negative cells responded with readily detectable growth in response to ligation of the BCR and even more so in response to SAC as well as to cytokine stimulation (FIG. 13). In contrast, FcRH4-positive cells responded well to cytokine stimulation but showed virtually no growth response to BCR ligation (FIG. 13).

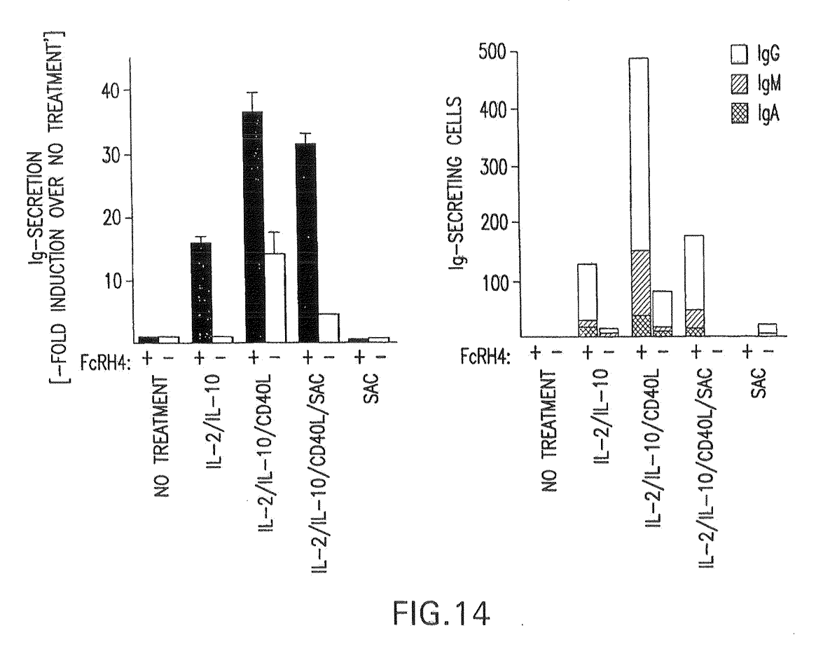

Studies by various groups have established that memory B cells can be induced to secrete immunoglobulins in vitro (Tangye, S. G., et al., J Immunol 170, 261-9 (2003); Agematsu, K. et al., Blood 91, 173-80 (1998)). Stimuli that induce Ig secretion are the T cell derived cytokines IL-2, IL-4 and IL-10 as well as ligation of CD40 by CD40L. We analyzed the secretion of total Ig from purified FcRH4-positive and FcRH4-negative memory B cells in response to cytokine stimulation with or without SAC. Analysis of the culture supernatant 4 days after cytokine addition revealed that FcRH4-positive cells secreted more Ig after treatment with IL-2/IL-10 and IL-2/IL-10/CD40L or IL-2/IL-10/CD40L/SAC than FcRH4-negative cells (FIG. 14A). Elispot assays of those cells revealed that the increased immunoglobulin secretion correlated with an increased number of Ig secreting cells as opposed to an equal number of Ig secreting B cells with increased amounts of Ig-secretion per cell by FcRH4-positive cells (FIG. 14B).

The possibility that the FcRH4⁻ memory B cells could be induced to express FcRH4⁻ as an intermediate step in memory B cell differentiation was also examined. FcRH4⁻ cells were labeled with a succinimidyl ester of carboxyfluorescein diacetate (CFSE), a fluorescent dye that is equally distributed between daughter cells. The stimulation of FcRH4⁻ memory B cells with IL-2, IL-10, and CD40L led to their proliferation, but this response was not accompanied by the expression of FcRH4 within the 48-h interval of observation.

### Example 3

To determine if FcRHs are expressed by peripheral blood derived B-CLL cells, flow cytometry was performed using mAbs to CD19, CD5, FcRH1, and CD38. Staining of four patient samples (detailed in Table 5) revealed no CD38 expression in the CD19+CD5+ populations analyzed, but a moderate level of FcRH1 expression was seen on all samples.

TABLE 5

| | Clinical | Months from | | Mutation | CDR3 |
|---|---|---|---|---|---|
| Sample | Course | Therapy | VH Gene | Status | Length |
| 61F | Treated | 10 | V 1-46 | 90% | 16aa |
| 62F | Stable | NA | V 1-69 | 88%* | — |
| 66F | Treated | 2 | V 4-34 | 98% | 18aa |
| 68F | Stable | NA | V 1-69 | 91% | 17aa |

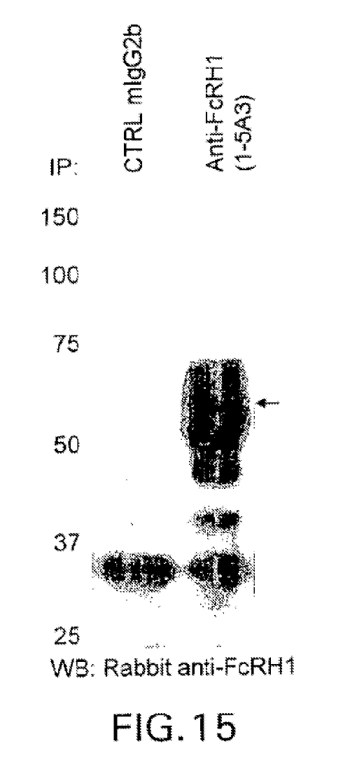

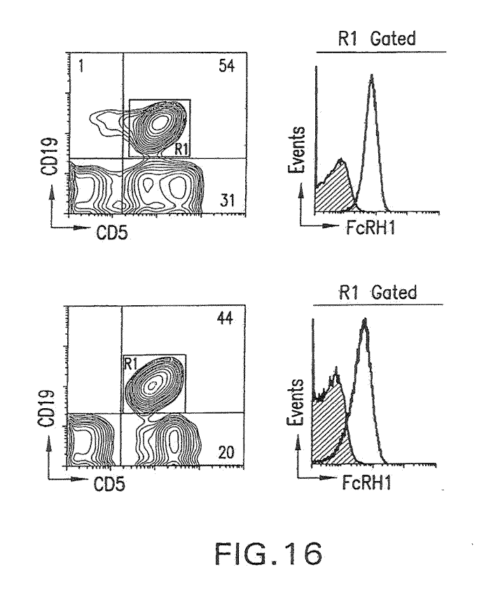

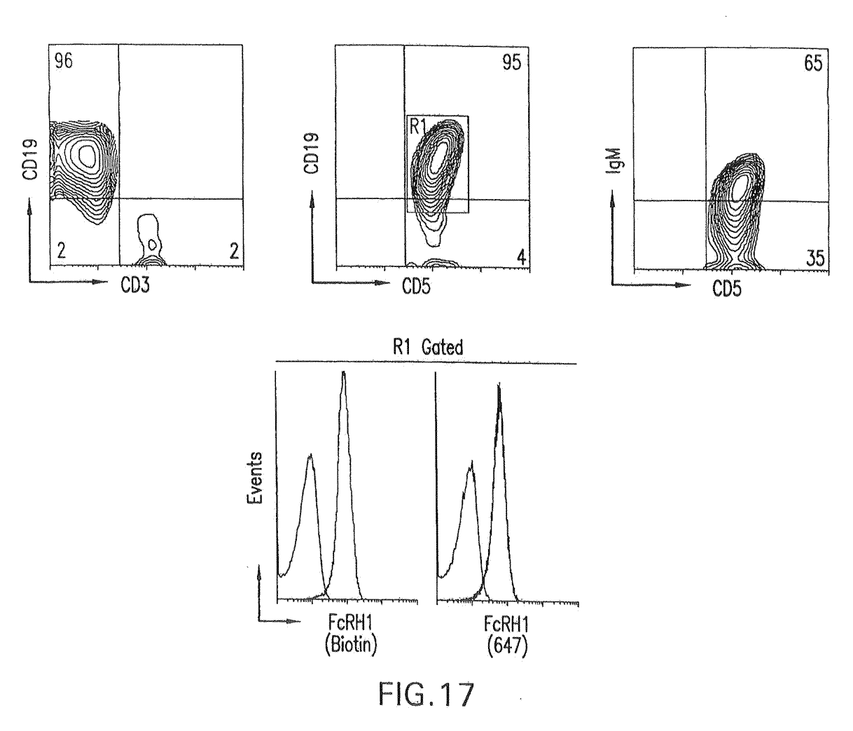

To evaluate if FcRH1 could be immunoprecipitated from B-CLL cells, whole cell lysate (WCL) was prepared from leukemic cells with the mutated IgVH genotype (VH 3-53 94% germline), immunoprecipitated with a mouse anti-FcRH1 mAb (1-5A3/mouse γ2bκ) or control (mouse γ2bκ), and immunoblotted with rabbit anti-FcRH1 polyclonal antiserum (FIG. 15). 5×10⁷ cells were lysed in 1% NP-40 lysis buffer, and incubated with the indicated mAbs before immunoprecipitation (IP) with Protein G beads. Eluted material was resolved by 7.5% SDS-PAGE under reducing conditions, immunoblotted (WB) with rabbit anti-FcRH1 antiserum and goat anti-rabbit horse-radish peroxidase, and visualized by enhanced chemiluminescence. Note the Mr of FcRH1 is ˜58 KDa. FcRH1 was identified in abundance from the B-CLL sample. FcRH1 can be detected by flow cytometry (n=6) and by biochemical immunoprecipitation (n=1) from B-CLL cells. These results indicate that FcRH1 is abundantly expressed in patients with B-CLL. FIG. 16 further demonstrates the expression of FcRH1 in B-CLL cells. Moreover, FIG. 17 demonstrates expression of FcRH 1 in mantle cell lymphoma.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

Ravetch J V, Kinet J P. Fc receptors. Annu. Rev. Immunol. 1991; 9:457-492.
Daeron M. Fc receptor biology. Annu. Rev. Immunol. 1997; 15:203-234.
Vely F, Vivier E. Conservation of structural features reveals the existence of a large family of inhibitory cell surface receptors and noninhibitory/activatory counterparts. J. Immunol. 1997; 159: 2075-2077.
Davis R S, Wang Y H, Kubagawa H, Cooper M D. Identification of a family of immunoglobulin Fc Receptor homologs with preferential B cell expression. Proc. Natl. Acad. Sci. 2001; 98: 9772-9777.
Miller I, Hatzivassiliou G, Cattoretti G, Mendelsohn C, Dalla-Favera R. IRTAs: a new family of immunoglobulin-like receptors differentially expressed in B cells. Blood 2002; 99: 2662-2669.
Pascual V, Liu Y J, Magalski A, de Bouteiller O, Banchereau J, Capra JD. Analysis of somatic mutation in five B cell subsets of human tonsil. J. Exp. Med. 1994; 180: 329-339.
Alizadeh, A. A., et al., Distinct types of diffuse large B-cell lymphoma identified by gene expression profiling. Nature 2000; 430: 503-511.
Reff M. E., Hariharan, K., Braslawsky, G., Future of Monoclonal Antibodies in the Treatment of Hematologic Malignancies. 2002; 9(2): 152-166.
Ehrhardt, G. R. et al. The inhibitory potential of Fc receptor homolog 4 on memory B cells. Proc Natl Acad Sci U S A 100, 13489-94 (2003).
Ehrhardt, G. R., Leslie, K. B., Lee, F., Wieler, J. S. & Schrader, J. W. M-Ras, a widely expressed 29-kD homologue of p21 Ras: expression of a constitutively active mutant results in factor-independent growth of an interleukin-3-dependent cell line. Blood 94, 2433-44 (1999).
Lefranc, M. P. IMGT, the international ImMunoGeneTics database. Nucleic Acids Res 31, 307-10 (2003).
Bohnhorst, J. O., Bjorgan, M. B., Thoen, J. E., Natvig, J. B. & Thompson, K. M. Bm1-Bm5 classification of peripheral blood B cells reveals circulating germinal center founder cells in healthy individuals and disturbance in the B cell subpopulations in patients with primary Sjogren's syndrome. J Immunol 167, 3610-8 (2001).
Pascual, V. et al. Analysis of somatic mutation in five B cell subsets of human tonsil. J Exp Med 180, 329-39 (1994).
Ellyard, J. I. et al. Antigen-selected, immunoglobulin-secreting cells persist in human spleen and bone marrow. Blood 103, 3805-12 (2004).
Shapiro-Shelef, M. et al. Blimp-1 is required for the formation of immunoglobulin secreting plasma cells and pre-plasma memory B cells. Immunity 19, 607-20 (2003).
Mittrucker, H. W. et al. Requirement for the transcription factor LSIRF/IRF4 for mature B and T lymphocyte function. Science 275, 540-3 (1997).
Galibert, L. et al. Negative selection of human germinal center B cells by prolonged BCR cross-linking. J Exp Med 183, 2075-85 (1996).
Tangye, S. G., Avery, D. T. & Hodgkin, P. D. A division-linked mechanism for the rapid generation of Ig-secreting cells from human memory B cells. J Immunol 170, 261-9 (2003).
Agematsu, K. et al. Generation of plasma cells from peripheral blood memory B cells: synergistic effect of interleukin-10 and CD27/CD70 interaction. Blood 91, 173-80 (1998).
Iwakoshi, N. N. et al. Plasma cell differentiation and the unfolded protein response intersect at the transcription factor XBP-1. Nat Immunol 4, 321-9 (2003).

What is claimed is:

1. An antibody having the same epitope specificity as an antibody produced by the hybridoma cell line deposited under the American Type Culture Collection Accession Number ATCC # PTA-6236.

2. An antibody produced by the hybridoma cell line deposited under the American Type Culture Collection Accession Number ATCC # PTA-6236.

3. A fragment of the antibody of claim 1, wherein the fragment binds to an FcRH4 receptor molecule.

4. A fragment of the antibody of claim 3, wherein the fragment activates the FcRH4 receptor molecule.

5. A molecular complex comprising the antibody of claim 1 and a therapeutic agent.

6. A molecular complex comprising the antibody fragment of claim 3 and a therapeutic agent.

7. A humanized version of the antibody of claim 1, wherein the humanized version binds to an FcRH4 receptor molecule.

8. An antibody of claim 7, wherein the antibody activates the FcRH4 receptor molecule.

9. The antibody of claim 1, wherein the antibody is labeled with a detectable moiety.

10. The antibody fragment of claim 3, wherein the antibody fragment is labeled with a detectable moiety.

11. The antibody of claim 7, comprising at least one complementarity determining region
(CDR) of the antibody of claim 1.

12. The antibody of claim 11, comprising all complementarity determining regions (CDRs) of the antibody of claim 1.

13. A molecular complex comprising the antibody of claim 7 and a therapeutic agent.

14. A human antibody that binds to the same epitope of an FcRH4 receptor molecule as the monoclonal antibody produced by the hybridoma cell line deposited under the American Type Culture Collection Accession Number ATCC # PTA-6236.

15. The human antibody of claim 14 wherein the antibody comprises at least one complementarity determining region (CDR) of the antibody of claim 1.

16. The antibody of claim 15, comprising all complementarity determining regions (CDR) of the antibody of claim 1.

17. A fragment of the antibody of claim 14, wherein the fragment binds to an FcRH4 receptor.

18. The antibody fragment of claim 17, wherein the antibody fragment activates the FcRH4 receptor molecule.

19. A molecular complex comprising the antibody of claim 14 and a therapeutic agent.

20. A molecular complex comprising the antibody fragment of claim 18 and a therapeutic agent.

21. A hybridoma cell that produces the antibody of claim 1.

22. A cell of the hybridoma cell line deposited under the American Type Culture Collection Accession Number ATCC # PTA-6236.

23. A method of diagnosing a malignancy of hematopoietic cell lineage in a subject, comprising:
a) contacting a biological sample of the subject with the antibody of claim 1 or fragment thereof under conditions that allow the antibody or fragment to bind to FcRH4 in the biological sample;
b) detecting binding by the antibody or fragment, changes in the binding as compared to binding in a control sample indicating a malignancy of hematopoietic cell lineage in the subject.

24. The method of claim 23, wherein the malignancy of hematopoietic cell lineage is a malignancy of B cell lineage.

25. The method of claim 23, wherein the malignancy of hematopoietic cell lineage is a malignancy of T cell lineage.

26. A method of identifying a hematopoietic cell that expresses FcRH4 in vitro, comprising:
a) contacting a biological sample with the antibody of claim 1 or fragment thereof under conditions that allow the antibody or fragment to bind to FcRH4 in the sample;
b) detecting the binding of the antibody or fragment, the binding identifying a hematopoietic cell that expresses FcRH4.

27. A method of selecting a hematopoietic cell that expresses FcRH4 or a purified population of hematopoietic cells that express FcRH4, comprising:
a) contacting a biological sample with the antibody of claim 1 or fragment thereof under conditions that allow the antibody or fragment to bind to FcRH4 expressing cells in the biological sample; and
b) selecting the cell or cells that bind the antibody or fragment, the selected cells being a hematopoietic cell that expresses FcRH4.

28. A method of treating a subject with a malignancy of a hematopoietic cell lineage, comprising administering to the subject a therapeutically effective amount of the antibody of claim 1 or a fragment thereof.

29. The method of claim 28, wherein the malignancy is selected from the group consisting of B-cell chronic lymphocytic leukemia (B-CLL), diffuse large B-Cell lymphomas, follicular lymphomas, mantle cell lymphomas, mucosa-associated lymphoid tissue (MALT) lymphomas, multiple myeloma, and Waldenstrom's macroglobulinemia.

30. The method of claim 28, wherein the malignancy of hematopoietic cell lineage is a malignancy of B cell lineage.

31. The method of claim 28, wherein the malignancy of hematopoietic cell lineage is a malignancy of T cell lineage.

32. A method of treating a subject with a malignancy of hematopoietic cell lineage, comprising contacting a malignant cell of the subject with a therapeutically effective amount of the molecular complex of claim 5.

33. The method of claim 32, wherein the malignancy is selected from the group consisting of B-cell chronic lymphocytic leukemia (B-CLL), diffuse large B-Cell lymphomas, follicular lymphomas, mantle cell lymphomas, mucosa-associated lymphoid tissue (MALT) lymphomas, multiple myeloma, and Waldenstrom's macroglobulinemia.

34. A method of diagnosing an autoimmune disease in a subject, comprising:
a) contacting a biological sample of the subject with the antibody of claim 1 or a fragment thereof under conditions that allow the antibody or fragment to bind to FcRH4 in the biological sample;
b) detecting binding by the antibody or fragment, changes in the antibody binding as compared to binding in a control sample indicating an autoimmune disease in the subject.

35. A method of treating an autoimmune disease in a subject, comprising contacting, with a therapeutically effective amount of the antibody of claim 1 or a fragment thereof, one or more FcRH4 expressing cells of the subject.

36. A method of treating an autoimmune disease in a subject, comprising contacting, with a therapeutically effective amount of the molecular complex of claim 5, one or more FcRH4 expressing cells of the subject.

37. A method of modulating a humoral immune response in a subject, comprising administering to the subject the antibody of claim 1 or a fragment thereof.

38. The method of claim 37, wherein the humoral immune response is enhanced.

39. The method of claim 38, wherein the humoral immune response is enhanced in a subject with an immunodeficiency.

40. The method of claim 38, wherein the humoral immune response is enhanced in a subject with an infectious disease.

41. The method of claim 37, wherein the humoral immune response is reduced.

42. A method of targeting B cells in a subject comprising administering to the subject the antibody of claim 1 or a fragment thereof.

43. The method of claim 42 further comprising administering to the subject a therapeutic agent that binds the antibody of claim 1 or a fragment thereof.

44. A method of targeting B cells in a subject comprising administering to the subject the complex of claim 5.

45. A polypeptide comprising one or more complementarity determining regions (CDRs) of the antibody of claim 1.

46. A nucleic acid that encodes the polypeptide of claim 45.

47. A polypeptide comprising one or more complementarity determining regions (CDRs) of the antibody of claim 1 with one or more conservative amino acid substitutions.

48. A nucleic acid that encodes the polypeptide of claim 47.

49. A fragment of the antibody of claim 1, wherein the fragment inhibits the FcRH4 receptor molecule.

50. An antibody having the same epitope specificity as an antibody produced by the hybridoma cell line deposited under the American Type Culture Collection Accession Number ATCC # PTA-6192.

51. An antibody produced by the hybridoma cell line deposited under American Type Culture Collection Accession Number ATCC # PTA-6192.

52. A fragment of the antibody of claim 50, wherein the fragment binds to an FcRH1 receptor molecule.

53. A fragment of the antibody of claim 52, wherein the fragment activates the FcRH1 receptor molecule.

54. A molecular complex comprising the antibody of claim 50 and a therapeutic agent.

55. A molecular complex comprising the antibody fragment of claim 52 and a therapeutic agent.

56. A humanized version of the antibody of claim 50, wherein the humanized version binds to an FcRH1 receptor molecule.

57. An antibody of claim 56, wherein the antibody activates the FcRH1 receptor molecule.

58. The antibody of claim 50, wherein the antibody is labeled with a detectable moiety.

59. The antibody fragment of claim 52, wherein the antibody fragment is labeled with a detectable moiety.

60. The antibody of claim 56, comprising at least one complementarity determining region (CDR) of the antibody of claim 50.

61. The antibody of claim 60, comprising all complementarity determining regions (CDRs) of the antibody of claim 50.

62. A molecular complex comprising the antibody of claim 50 and a therapeutic agent.

63. A human antibody that binds to the same epitope of an FcRH1 receptor molecule as the monoclonal antibody produced by the hybridoma cell line deposited under American Type Culture Collection Accession Number ATCC # PTA-6192.

64. The human antibody of claim 63, wherein the antibody comprises at least one complementarity determining region (CDR) of the antibody of claim 50.

65. The antibody of claim 64, comprising all complementarity determining regions (CDR) of the antibody of claim 50.

66. A fragment of the antibody of claim 63, wherein the fragment binds to an FcRH1 receptor.

67. The antibody fragment of claim 66, wherein the antibody fragment activates the FcRH1 receptor molecule.

68. A molecular complex comprising the antibody of claim 63 and a therapeutic agent.

69. A molecular complex comprising the antibody fragment of claim 67 and a therapeutic agent.

70. A hybridoma cell that produces the antibody of claim 50.

71. A cell of the hybridoma cell line deposited under American Type Culture Collection Accession Number ATCC # PTA-6192.

72. A method of diagnosing a malignancy of hematopoietic cell lineage in a subject, comprising:
a) contacting a biological sample of the subject with the antibody of claim 50 or fragment thereof under conditions that allow the antibody or fragment to bind to FcRH1 in the biological sample;
b) detecting binding by the antibody or fragment, changes in the binding as compared to binding in a control sample indicating a malignancy of hematopoietic cell lineage in the subject.

73. The method of claim 72, wherein the malignancy of hematopoietic cell lineage is a malignancy of 13 cell lineage.

74. The method of claim 72, wherein the malignancy of hematopoietic cell lineage is a malignancy of T cell lineage.

75. A method of identifying a hematopoietic cell that expresses FcRH1 in vitro, comprising:
a) contacting a biological sample with the antibody of claim 50 or fragment thereof under conditions that allow the antibody or fragment to bind to FcRH1 in the sample;
b) detecting the binding of the antibody or fragment, the binding identifying a hematopoietic cell that expresses FcRH1.

76. A method of selecting a hematopoietic cell that expresses FcRH1 or a purified population of hematopoietic cells that express FcRH1, comprising:
a) contacting a biological sample with the antibody of claim 50 or fragment thereof under conditions that allow the antibody or fragment to bind to FcRH1 expressing cells in the biological sample; and
b) selecting the cell or cells that bind the antibody or fragment, the selected cells being a hematopoietic cell that expresses FcRH1.

77. A method of treating a subject with a malignancy of a hematopoietic cell lineage, comprising administering to the subject a therapeutically effective amount of the antibody of claim 50 or a fragment thereof.

78. The method of claim 77, wherein the malignancy is selected from the group consisting of B-cell chronic lymphocytic leukemia (B-CLL), diffuse large B-Cell lymphomas, follicular lymphomas, mantle cell lymphomas, mucosa-associated lymphoid tissue (MALT) lymphomas, multiple myeloma, and Waldenstrom's macroglobulinemia.

79. The method of claim 77, wherein the malignancy of hematopoietic cell lineage is a malignancy of B cell lineage.

80. The method of claim 77, wherein the malignancy of hematopoietic cell lineage is a malignancy of T cell lineage.

81. A method of treating a subject with a malignancy of hematopoietic cell lineage, comprising contacting a malignant cell of the subject with a therapeutically effective amount of the molecular complex of claim 54.

82. The method of claim 81, wherein the malignancy is selected from the group consisting of B-cell chronic lymphocytic leukemia (B-CLL), diffuse large B-Cell lymphomas, follicular lymphomas, mantle cell lymphomas, mucosa-associated lymphoid tissue (MALT) lymphomas, multiple myeloma, and Waldenstrom's macroglobulinemia.

83. A method of diagnosing an autoimmune disease in a subject, comprising:
a) contacting a biological sample of the subject with the antibody of claim 50 or a fragment thereof under conditions that allow the antibody or fragment to bind to FcRH1 in the biological sample;
b) detecting binding by the antibody or fragment, changes in the antibody binding as compared to binding in a control sample indicating an autoimmune disease in the subject.

84. A method of treating an autoimmune disease in a subject, comprising contacting, with a therapeutically effective amount of the antibody of claim 50 or a fragment thereof, one or more FcRH1 expressing cells of the subject.

85. A method of treating an autoimmune disease in a subject, comprising contacting, with a therapeutically effective amount of the molecular complex of claim 54, one or more FcRH1 expressing cells of the subject.

86. A method of modulating a humoral immune response in a subject, comprising administering to the subject the antibody of claim 50 or a fragment thereof.

87. The method of claim 86, wherein the humoral immune response is enhanced.

88. The method of claim 87, wherein the humoral immune response is enhanced in a subject with an immunodeficiency.

89. The method of claim 87, wherein the humoral immune response is enhanced in a subject with an infectious disease.

90. The method of claim 86, wherein the humoral immune response is reduced.

91. A method of targeting B cells in a subject comprising administering to the subject the antibody of claim 50 or a fragment thereof.

92. The method of claim 91 further comprising administering to the subject a therapeutic agent that binds the antibody of claim 50 or a fragment thereof.

93. A method of targeting B cells in a subject comprising administering to the subject the complex of claim 54.

94. A polypeptide comprising one or more complementarity determining regions (CDRs) of the antibody of claim 50.

95. A nucleic acid that encodes the polypeptide of claim 94.

96. A polypeptide comprising one or more complementarity determining regions (CDRs) of the antibody of claim 50 with one or more conservative amino acid substitutions.

97. A nucleic acid that encodes the polypeptide of claim 96.

98. A fragment of the antibody of claim 52, wherein the fragment inhibits the FcRH1 receptor molecule.

99. An antibody having the same epitope specificity as an antibody produced by the hybridoma cell line deposited under the American Type Culture Collection Accession Number ATCC # PTA-6219.

100. An antibody produced by the hybridoma cell line deposited under the American Type Culture Collection Accession Number ATCC # PTA-6219.

101. A fragment of the antibody of claim 99, wherein the fragment binds to an FcRH1 receptor molecule.

102. A fragment of the antibody of claim 101, wherein the fragment activates the FcRH1 receptor molecule.

103. A molecular complex comprising the antibody of claim 99 and a therapeutic agent.

104. A molecular complex comprising the antibody fragment of claim 101 and a therapeutic agent.

105. A humanized version of the antibody of claim 99, wherein the humanized version binds to an FcRH1 receptor molecule.

106. An antibody of claim 105, wherein the antibody activates the FcRH1 receptor molecule.

107. The antibody of claim 99, wherein the antibody is labeled with a detectable moiety.

108. The antibody fragment of claim 101, wherein the antibody fragment is labeled with a detectable moiety.

109. The antibody of claim 105, comprising at least one complementarity determining region (CDR) of the antibody of claim 99.

110. The antibody of claim 109, comprising all complementarity determining regions (CDRs) of the antibody of claim 99.

111. A molecular complex comprising the antibody of claim 105 and a therapeutic agent.

112. A human antibody that binds to the same epitope of an FcRH1 receptor molecule as the monoclonal antibody produced by the hybridoma cell line deposited under the American Type Culture Collection Accession Number ATCC # PTA-6236.

113. The human antibody of claim 112 wherein the antibody comprises at least one complementarity determining region (CDR) of the antibody of claim 99.

114. The antibody of claim 113, comprising all complementarity determining regions (CDR) of the antibody of claim 99.

115. A fragment of the antibody of claim 112, wherein the fragment binds to an FcRH1 receptor.

116. The antibody fragment of claim 115, wherein the antibody fragment activates the FcRH1 receptor molecule.

117. A molecular complex comprising the antibody of claim 112 and a therapeutic agent.

118. A molecular complex comprising the antibody fragment of claim 116 and a therapeutic agent.

119. A hybridoma cell that produces the antibody of claim 99.

120. A cell of the hybridoma cell line deposited under the American Type Culture Collection Accession Number ATCC # PTA-6219.

121. A method of diagnosing a malignancy of hematopoietic cell lineage in a subject, comprising:
a) contacting a biological sample of the subject with the antibody of claim 99 or fragment thereof under conditions that allow the antibody or fragment to bind to FcRH1 in the biological sample;
b) detecting binding by the antibody or fragment, changes in the binding as compared to binding in a control sample indicating a malignancy of hematopoietic cell lineage in the subject.

122. The method of claim 121, wherein the malignancy of hematopoietic cell lineage is a malignancy of B cell lineage.

123. The method of claim 121, wherein the malignancy of hematopoietic cell lineage is a malignancy of T cell lineage.

124. A method of identifying a hematopoictic cell that expresses FcRH1 in vitro, comprising:
a) contacting a biological sample with the antibody of claim 99 or fragment thereof under conditions that allow the antibody or fragment to bind to FcRH1 in the sample;
b) detecting the binding of the antibody or fragment, the binding identifying a hematopoietic cell that expresses FcRH1.

125. A method of selecting a hematopoietic cell that expresses FcRH1 or a purified population of hematopoietic cells that express FcRH1, comprising:
a) contacting a biological sample with the antibody of claim 99 or fragment thereof under conditions that allow the antibody or fragment to bind to FcRH1 expressing cells in the biological sample; and
b) selecting the cell or cells that bind the antibody or fragment, the selected cells being a hematopoietic cell that expresses FcRH1.

126. A method of treating a subject with a malignancy of a hematopoietic cell lineage, comprising administering to the subject a therapeutically effective amount of the antibody of claim 99 or a fragment thereof.

127. The method of claim 126, wherein the malignancy is selected from the group consisting of B-cell chronic lymphocytic leukemia (B-CLL), diffuse large B-Cell lymphomas, follicular lymphomas, mantle cell lymphomas, mucosa-associated lymphoid tissue (MALT) lymphomas, multiple myeloma, and Waldenstrom's macroglobulinemia.

128. The method of claim 126, wherein the malignancy of hematopoietic cell lineage is a malignancy of B cell lineage.

129. The method of claim 126, wherein the malignancy of hematopoietic cell lineage is a malignancy of T cell lineage.

130. A method of treating a subject with a malignancy of hematopoietic cell lineage, comprising contacting a malignant cell of the subject with a therapeutically effective amount of the molecular complex of claim 103.

131. The method of claim 130, wherein the malignancy is selected from the group consisting of B-cell chronic lymphocytic leukemia (B-CLL), diffuse large B-Cell lymphomas, follicular lymphomas, mantle cell lymphomas, mucosa-associated lymphoid tissue (MALT) lymphomas, multiple myeloma, and Waldenstrom's macroglobulinemia.

132. A method of diagnosing an autoimmune disease in a subject, comprising:
a) contacting a biological sample of the subject with the antibody of claim 99 or a fragment thereof under conditions that allow the antibody or fragment to bind to FcRH1 in the biological sample;
b) detecting binding by the antibody or fragment, changes in the antibody binding as compared to binding in a control sample indicating an autoimmune disease in the subject.

133. A method of treating an autoimmune disease in a subject, comprising contacting, with a therapeutically effective amount of the antibody of claim 99 or a fragment thereof, one or more FcRH1 expressing cells of the subject.

134. A method of treating an autoimmune disease in a subject, comprising contacting, with a therapeutically effective amount of the molecular complex of claim 103, one or more FcRH1 expressing cells of the subject.

135. A method of modulating a humoral immune response in a subject, comprising administering to the subject the antibody of claim 99 or a fragment thereof.

136. The method of claim 135, wherein the humoral immune response is enhanced.

137. The method of claim 136, wherein the humoral immune response is enhanced in a subject with an immunodeficiency.

138. The method of claim 136, wherein the humoral immune response is enhanced in a subject with an infectious disease.

139. The method of claim 135, wherein the humoral immune response is reduced.

140. A method of targeting B cells in a subject comprising administering to the subject the antibody of claim 99 or a fragment thereof.

141. The method of claim 140 further comprising administering to the subject a therapeutic agent that binds the antibody of claim 99 or a fragment thereof.

142. A method of targeting B cells in a subject comprising administering to the subject the complex of claim 103.

143. A polypeptide comprising one or more complementarity determining regions (CDRs) of the antibody of claim 99.

144. A nucleic acid that encodes the polypeptide of claim 143.

145. A polypeptide comprising one or more complementarity determining regions (CDRs) of the antibody of claim 99 with one or more conservative amino acid substitutions.

146. A nucleic acid that encodes the polypeptide of claim 145.

147. A fragment of the antibody of claim 99, wherein the fragment inhibits the FcRH1 receptor molecule.

148. A method of identifying an agent that alters modulation of the FcRH1 receptor comprising:
a) contacting a cell comprising the FcRH1 receptor with a test agent in the presence of the antibody of claim 50 or a fragment thereof; and
b) determining the level of modulation of the FcRH1 receptor as compared to a control cell comprising the FcRH1 receptor contacted with the antibody of claim 50 or a fragment thereof in the absence of the test agent, a difference in modulation indicating that the test agent alters modulation of the FcRH1 receptor by the antibody of claim 50 or a fragment thereof.

149. A method of identifying an agent that alters modulation of the FcRH4 receptor comprising:
a) contacting a cell comprising the FcRH4 receptor with a test agent in the presence of the antibody of claim 1 or a fragment thereof; and
b) determining the level of modulation of the FcRH4 receptor as compared to a control cell comprising the FcRH4 receptor contacted with the antibody of claim 1 or a fragment thereof in the absence of the test agent, a difference in modulation indicating that the test agent alters modulation of the FcRH4 receptor by the antibody of claim 1 or a fragment thereof.
